(19) United States
(12) Patent Application Publication
(10) Pub. No.: US 2017/0326044 A1
(43) Pub. Date: Nov. 16, 2017

(54) COSMETIC COMPOSITION COMPRISING A SYNTHETIC PHYLLOSILICATE AND AN ELECTROLYTE AND/OR A POLYELECTROLYTE

(71) Applicant: L'OREAL, Paris (FR)

(72) Inventors: Fabrice SPRINGINSFELD, Chevilly-Larue (FR); Chantal JOUY, Chevilly-Larue (FR); Mathieu CHABRILLANGEAS, Chevilly-Larue (FR); Laurence ARNAUD-SEBILLOTTE, Chevilly-Larue (FR); Raluca LORANT, Chevilly-Larue (FR); Nathalie JAGER LEZER, Chevilly-Larue (FR)

(73) Assignee: L'OREAL, Paris (FR)

(21) Appl. No.: 15/529,417

(22) PCT Filed: Nov. 24, 2015
(86) PCT No.: PCT/EP2015/077569

§ 371 Date: May 24, 2017

(30) Foreign Application Priority Data
Nov. 24, 2014 (FR) 1461347
Nov. 24, 2014 (FR) 1461356

(51) Int. Cl. A61K 8/25; A61K 8/73; A61K 8/28; A61K 8/26; A61K 8/365; A61Q 19/00; A61K 8/81

(52) U.S. Cl. CPC A61K 8/25; A61Q 19/00; A61K 8/73; A61K 8/8147; A61K 8/26; A61K 8/365; A61K 8/28; A61K 2800/10

(57) ABSTRACT

The present invention relates to a composition, in particular cosmetic, comprising: (a) at least one synthetic phyllosilicate with molecular formula Mg3Si4O10(OH)2 in the form of an aqueous or hydroalcoholic gel; and (b) at least one electrolyte and/or at least one polyelectrolyte. The present invention also relates to a method for cosmetic treatment of the skin and/or nails, comprising the application of said composition to the skin and/or the nails, as well as a cosmetic method for treating perspiration and possibly body odour linked to human perspiration, in particular odours from the armpits and the feet, characterised in that it consists of applying said composition to the surface of the skin and, specifically, to the armpits and/or the feet.

The present invention relates to compositions, especially cosmetic compositions, preferably in the form of direct or inverse emulsions, preferably for topical application, comprising electrolytes and/or polyelectrolytes. More specifically, the invention relates to the field of caring for, making up and hygiene of the skin and/or the nails, and particularly bodily skin.

The term “skin” is intended to mean facial and/or bodily skin. Preferably, said skin will be facial and/or bodily skin.

The term “nails” also means false nails insofar as the desired cosmetic effects are often identical.

It is common practice in cosmetics to use topical compositions in the form of aqueous gels or emulsions. Gelling agents or thickeners are incorporated therein to adjust the consistency and the texture of these compositions. The reason for this is that the consistency and above all the texture of the compositions condition not only the cosmetic feel, but also the attraction of consumers toward the product.

The presence of gelling agents or thickeners in compositions, especially cosmetic compositions, may also be a determining factor for stabilizing the emulsions and/or dispersions of solid particles such as fillers and pigments.

As illustrations of gelling agents (or thickeners) that are conventionally used, mention may especially be made of:
linear or branched, hydrophobic or non-hydrophobic acrylic polymers;
linear or branched, hydrophobic or non-hydrophobic acrylamide polymers;
linear or branched, hydrophobic or non-hydrophobic polymers derived from acrylamidomethylpropanesulfonic acid derivatives; and
hydrophobic or non-hydrophobic polymers of natural origin such as celluloses, xanthans, guars, alginates and gelatins.

In parallel, certain cosmetic compositions of emulsion or thickened architecture are devoted to conveying active agents in the form of electrolytes or polyelectrolytes, and of doing so occasionally even in a relatively large amount.

Unfortunately, these electrolytes and polyelectrolytes are not always compatible, this being the case with the majority of the gelling agents considered in cosmetic compositions.

It is known, for example, that electrolytes are incompatible with neutralized carboxyvinyl polymers, which is reflected by a strong decrease in the viscosity of the gels obtained from these polymers; this results in destabilization of the emulsion, and may be reflected by phase separation. Thus, compositions containing carboxyvinyl polymers and electrolytes lack consistency, which is contrary to the desired result for the use of a gelling agent.

Among the abovementioned polymeric gelling agents, hydrophobic polymers are the ones that best resist electrolytes and polyelectrolytes. However, the viscosity of the composition decreases during the addition of the electrolytes and polyelectrolytes, and readjustment of the viscosity by adding a hydrophobic polymer proves to be prejudicial in terms of cost.

Another solution for gelling or thickening cosmetic compositions comprising electrolytes and/or polyelectrolytes is to make use of mineral particles such as laponites (lithium magnesium sodium silicate), bentonites (aluminum phyllosilicate) and montmorillonites (sodium calcium aluminum magnesium silicate hydroxide of formula (Na,Ca)_{0.33}(Al,Mg)₂(Si₄O₁₀)(OH)₂.nH₂O).

However, these mineral particles have certain drawbacks.

Specifically, for example, laponite particles are of nanometric size and their best stability in terms of pH is between 9.5 and 10, which makes them incompatible with cosmetic application.

In addition, natural mineral particles run the risk of being contaminated with heavy metals, asbestos, chlorite, etc., which, for obvious reasons, is not recommended for a use in the cosmetic field.

There is thus still a need to propose cosmetic compositions that are capable of conveying electrolytes and/or polyelectrolytes, in large amounts, while at the same time remaining stable, which have a satisfactory viscosity appreciated by consumers, and which are free of any risk of undesirable mineral contamination.

The objective of the present invention is especially to satisfy this need.

Thus, one object of the invention is a composition, especially a cosmetic composition, comprising:
(a) at least one synthetic phyllosilicate of molecular formula Mg₃Si₄O₁₀(OH)₂ in the form of an aqueous or aqueous-alcoholic gel; and
(b) at least one electrolyte and/or at least one polyelectrolyte.

Advantageously, the composition according to the present invention comprising said phyllosilicate presents an X-ray diffraction line greater than 9.4 Å and less than or equal to 9.8 Å.

Advantageously, the composition according to the present invention comprising said phyllosilicate has an infrared absorption band at 7200 cm⁻¹ corresponding to the stretching vibration attributed to the silanol groups Si—OH at the edge of the phyllosilicate leaflets.

Advantageously, the composition according to the present invention comprising said phyllosilicate is characterized by the absence of an infrared absorption band at 7156 cm⁻¹. This band at 7156 cm⁻¹ corresponds to the vibration band of Mg₂FeOH.

The composition according to the present invention comprising said phyllosilicate also preferably has an infrared absorption band at 7184 cm⁻¹ corresponding to the 2v Mg₃OH stretching vibration.

It should be noted that in the presence of adsorbed water, for example residual water, a broad infrared absorption band is detectable and readily identifiable, for example at 5500 cm⁻¹.

According to an embodiment, the composition according to the invention is a cosmetic or dermatological composition comprising a physiologically acceptable medium.

Synthetic phyllosilicates such as those described in patent application WO 2008/009799 and advantageously those disclosed in patent application FR 2 977 580 are most particularly suitable for use in the invention.

However, neither of these documents WO2008/009799 or FR 2 977 580 considers exploiting the synthetic phyllosilicates thus obtained in compositions and especially for cosmetic, dermatological or pharmaceutical purposes.

In particular, neither of these documents considers a combination between these synthetic phyllosilicates and polyelectrolytes and/or electrolytes.

Now, contrary to all expectation, the inventors have found that the formulation of a synthetic phyllosilicate of molecular formula Mg₃Si₄O₁₀(OH)₂ in aqueous or aqueous-alcoholic gel form in compositions comprising at least one electrolyte and/or one polyelectrolyte as defined below makes it possible to give this system good stability, even at high concentrations of electrolytes and/or polyelectrolyte, and to conserve satisfactory viscosity that is appreciated by consumers, while at the same time providing good cosmetic properties in terms of texture quality and homogeneity of the deposit.

Moreover, as emerges from the experimental section, the combination of a synthetic phyllosilicate of molecular formula Mg₃Si₄O₁₀(OH)₂ in aqueous or aqueous-alcoholic gel form that is suitable for use in the invention with at least one electrolyte and/or at least one polyelectrolyte, such as an aluminum salt, makes it possible to obtain gels with lower contents of synthetic phyllosilicate.

Thus, as emerges from examples 2 and 3, the sol-gel transition, i.e. the passage from the liquid state to the gel state of the synthetic phyllosilicate, may take place in the presence of 15% aluminum salt active material in an amount of 2% to 3% by weight of synthetic phyllosilicate active material instead of an amount of between 4% and 5% by weight of active material in the absence of aluminum salt.

According to a particular embodiment, when the electrolyte is chosen from an aluminum and/or zirconium salt, an aluminum and/or zirconium complex, and mixtures thereof, preferably chosen from aluminum halohydrates, aluminum zirconium halohydrates, complexes of zirconium hydroxychloride and of aluminum hydroxychloride, with or without an amino acid, and mixtures thereof, a composition according to the invention may also comprise (c) at least one film-forming polymer chosen from a hydrophilic film-forming polymer and a hydrophobic film-forming polymer, and mixtures thereof.

Thus, according to a particular embodiment, the present invention relates to a composition, especially a cosmetic composition, comprising:
(a) at least one synthetic phyllosilicate in aqueous or aqueous-alcoholic gel form of molecular formula Mg₃Si₄O₁₀(OH)₂;
(b′) at least one compound chosen from an aluminum and/or zirconium salt, an aluminum and/or zirconium complex, and mixtures thereof;
(c) at least one film-forming polymer chosen from a hydrophilic film-forming polymer, a hydrophobic film-forming polymer, and mixtures thereof.

From this particular embodiment, the inventors have found that the formulation of a synthetic phyllosilicate in aqueous or aqueous-alcoholic gel form in compositions comprising at least one compound chosen from an aluminum and/or zirconium salt, an aluminum and/or zirconium complex, and mixtures thereof, and at least one film-forming polymer chosen from a hydrophilic film-forming polymer, a hydrophobic film-forming polymer, and mixtures thereof, makes it possible, in addition to the abovementioned advantages, to give this system good stability, and also an improvement in the quality of the film (especially the water resistance and the friction resistance) while at the same time giving good antiperspirant and deodorant efficacy.

Preferably, a composition according to the invention comprises at least one aluminum salt, preferably aluminum chlorohydrate.

Thus, according to a preferred mode of this particular embodiment, a composition according to the invention, especially a cosmetic composition, comprises:
(a) at least one synthetic phyllosilicate of molecular formula Mg₃Si₄O₁₀(OH)₂ in the form of an aqueous or aqueous-alcoholic gel;
(b″) at least one aluminum salt.
(c) at least one film-forming polymer chosen from a hydrophilic film-forming polymer, a hydrophobic film-forming polymer, and mixtures thereof.

As emerges from example 9 below, the compositions according to the invention make it possible to significantly reduce the white marks and yellow stains on fabrics (transfer-resistant nature) while at the same time improving the quality of the product deposit and its efficacy over time, and while giving good wettability with respect to the support, and also, in a persistent manner, resistance to moisture caused by perspiration and resistance to fat from sebum, and a dry feel, and while maintaining a pleasant sensory perception suitable for an antiperspirant/deodorant application.

The transfer-resistant nature of a composition corresponds to the fact that, once applied, it does not become appreciably deposited onto the surfaces with which it comes into contact, and more particularly clothing.

The invention is also directed toward a cosmetic process for treating perspiration and optionally the body odor associated with human perspiration and especially underarm odor and foot odor, characterized by the fact that it consists in applying to the surface of the skin, and more particularly to the armpits and/or the feet, a composition as defined previously.

The invention is also directed toward a cosmetic process for treating the skin and/or the nails, comprising the application, to the skin and/or the nails, of a composition as defined previously.

As explained in greater detail in the experimental section, a synthetic phyllosilicate that is suitable for use in the invention in gel form is not destructured during the addition of the electrolytes and/or polyelectrolytes and even contributes toward increasing the viscosity of the compositions comprising electrolytes and/or polyelectrolytes.

Thus, a synthetic phyllosilicate that is suitable for use in the invention makes it possible to give these compositions thickening or gelling power.

As detailed below, another advantage of a synthetic phyllosilicate that is suitable for use in the invention is that it is free of impurities or of undesirable compounds.

According to another advantage, a synthetic phyllosilicate that is suitable for use in the invention makes it possible to obtain good homogeneity of the deposit after application.

An additional advantage is the reduced cost of a synthetic phyllosilicate that is suitable for use in the invention when compared with the abovementioned hydrophobic polymers.

Synthetic Phyllosilicate

The synthetic phyllosilicate in accordance with the invention has a crystalline structure in accordance with that of a hydroxylated magnesium silicate of molecular formula Mg₃Si₄O₁₀(OH)₂ belonging to the chemical family of phyllosilicates.

These phyllosilicates are generally formed from a stack of elemental leaflets of crystalline structure, the number of which ranges from a few units to several tens of units. Each elemental leaflet is formed by the association of two layers of tetrahedra in which the silicon atoms are positioned, located on either side of a layer of octahedra in which the magnesium atoms are positioned. This group corresponds to the 2/1 phyllosilicates, which are also termed as being of T.O.T. (Tetrahedron-octahedron-tetrahedron) type.

As presented above, a synthetic phyllosilicate in accordance with the invention may be obtained according to a preparation process such as the one described in patent application WO 2008/009799 and is preferentially obtained according to the technology described in patent application FR 2 977 580.

This preparation process in particular comprises an extended hydrothermal treatment, which makes it possible to obtain an aqueous gel of synthetic phyllosilicate. Accordingly, according to a first embodiment, the synthetic phyllosilicate may be used in the form of an aqueous or aqueous-alcoholic gel, in particular like the one obtained directly on conclusion of the synthetic process.

As described in patent application FR 2 977 580, the parameters that influence the synthesis and the properties of a synthetic phyllosilicate in gel form that is suitable for use in the invention are the nature of the heat treatment (200° C. to 900° C.), the pressure, the nature of the reagents and the proportions thereof.

More particularly, the duration and temperature of the hydrothermal treatment make it possible to control the size of the particles. For example, the lower the temperature, the smaller the synthesized particles, as described in patent application FR 2 977 580. Controlling the size makes it possible to afford new properties and allows for better control of both its hydrophilic and hydrophobic properties, i.e. amphiphilic properties.

Structural Analysis and Characterization of a Synthetic Phyllosilicate that is Suitable for Use in the Invention

A synthetic phyllosilicate that is suitable for use in the invention may be characterized by various parameters, namely infrared absorption bands, its size and its purity, as detailed below.

Under certain conditions, analyses such as nuclear magnetic resonance in particular of ²⁹Si may be useful for the characterization of a synthetic phyllosilicate that is suitable for use in the invention. Similarly, thermogravimetric analysis (TGA) may be used for the characterization of a synthetic phyllosilicate that is suitable for use in the invention. Finally, X-ray diffraction may also be used for this purpose.

Infrared

Method Used

The machine used is a Nicolet 6700 FTIR Fourier transform spectrometer, equipped with an integration sphere, with an InGaA detector and a CaF₂ separator and a resolution of 12 cm⁻¹, more preferentially of 8 cm⁻¹ and even more preferentially of 4 cm⁻¹. In other words, the values of the infrared absorption bands given in this description should be considered as being more or less 6 cm⁻¹ and more preferentially more or less 4 cm⁻¹ and even more preferentially more or less 2 cm⁻¹.

The near infrared recordings of the stretching region located at 7184 cm⁻¹ were broken down by pseudo-Voigt functions using the Fityk software (Wojdyr, 2010).

To visualize the absorption spectrum in a composition comprising at least an aqueous part, such as an emulsion, it is recommended to heat this composition to a temperature corresponding to a temperature greater than or equal to 100° C. (for example 120° C.) and less than or equal to 500° C. (for example 400° C.) so as to eliminate the adsorbed water part and, where appropriate, some or all of the organic compound(s) present in the composition.

Generally, to confirm an infrared absorption band, a person skilled in the art performs stretching amplifications; in particular, they may, for example, perform such amplifications to plus or minus 200 cm⁻¹ on either side of a suspected infrared absorption band.

A natural talc is a mineral species composed of doubly hydroxylated magnesium silicate of formula Mg₃Si₄O₁₀(OH)₂, which may contain traces of nickel, iron, aluminum, calcium or sodium.

Natural talc has an infrared spectrum with a typical, fine and strong infrared absorption band at 7184 cm⁻¹ corresponding to the stretching vibration 2v Mg₃OH. Natural talc also contains chemical elements which replace magnesium and silicon in the crystalline structure, which impose the appearance of at least one additional infrared absorption band, in particular that corresponding to the stretching vibration at 7156 cm⁻¹ attributable to 2v Mg₂FeOH.

The spectrum of the synthetic phyllosilicate that is suitable for use in the invention differs from that of a natural talc by an infrared absorption band at 7200 cm⁻¹ corresponding to the stretching vibration attributed to the silanol groups Si—OH at the edge of the phyllosilicate leaflets.

To confirm this infrared absorption band, those skilled in the art may perform a stretching amplification, in particular in the region 7400 cm⁻¹-7000 cm⁻¹ and more particularly in the region 7300 cm⁻¹-7100 cm⁻¹.

Preferably, the spectrum of the synthetic phyllosilicate is also characterized by an absence of infrared absorption band at 7156 cm⁻¹. This band at 7156 cm⁻¹ corresponds to the vibration band of Mg₂FeOH.

Preferably, the spectrum of the synthetic phyllosilicate is also characterized by the infrared absorption band at 7184 cm⁻¹ which is common to natural talc.

It should be noted that in the presence of adsorbed water, for example residual water, a broad infrared absorption band is detectable and readily identifiable, for example at 5500 cm⁻¹.

Advantageously, the composition according to the present invention comprising said phyllosilicate has an infrared absorption band at 7200 cm⁻¹ corresponding to the stretching vibration attributed to the silanol groups Si—OH at the edge of the phyllosilicate sheets.

Advantageously, the composition according to the present invention comprising said phyllosilicate is characterized by the absence of an infrared absorption band at 7156 cm⁻¹. This band at 7156 cm⁻¹ corresponds to the vibration band of Mg₂FeOH.

The composition according to the present invention comprising said phyllosilicate also preferably has an infrared absorption band at 7184 cm⁻¹ corresponding to the 2v Mg₃OH stretching vibration.

In a composition according to the invention, it should be noted that in the presence of adsorbed water, for example residual water, a broad infrared absorption band is detectable and readily identifiable, for example at 5500 cm⁻¹.

Size

Method Used

In order to perform the particle size analysis of the synthetic phyllosilicates that are suitable for use in the invention, photon correlation spectroscopy was used. This analytical technique affords access to the size of the particles on the basis of the principle of dynamic light scattering. This device measures over time the intensity of the light scattered by the particles at an angle θ under consideration and the scattered rays are then processed using the Padé-Laplace algorithm.

This non-destructive technique requires dissolution of the particles. The particle size measurement obtained by this technique corresponds to the value of the hydrodynamic diameter of the particle, i.e. it comprises both the particle size and also the thickness of the hydration layer.

The analyses were performed using a VASC0-2 particle size analyzer from Cordouan. For the purpose of obtaining statistical information regarding the particle distribution, the NanoQ™ software was used in multi-acquisition mode with the Padé-Laplace algorithm.

Thus, a synthetic phyllosilicate that is suitable for use in the invention, in the form of an aqueous or aqueous-alcoholic gel, advantageously has a mean size ranging from 300 nm to 500 nm.

These characteristics are advantageous with regard to a natural talc, one of the constraints of which is the uncontrolled size of its particles.

Purity

The synthetic phyllosilicate under consideration according to the invention has a degree of purity of at least 99.90% and preferably of at least 99.99%.

It is thus advantageously free of impurities or of undesirable compounds, among which are in particular asbestos minerals such as asbestos (serpentine), chlorite, carbonates, heavy metals, iron sulfides, etc., which are generally associated with natural talc and/or incorporated into the structure of natural tales.

NMR (Nuclear Magnetic Resonance)

Methods Used

The silicon-29 (²⁹Si) NMR spectra were recorded on a Brüker Avance 400 (9.4 T) spectrometer. The reference for the chemical shifts is tetramethylsilane (TMS). The samples were placed in 4 mm zirconia rotors. The magic angle spin (MAS) speed was set at 8 kHz. The experiments were performed at a ambient temperature of 21° C.

The ²⁹Si spectra were obtained either by direct polarization (rotation of 30°) with a recycling delay of 60 s, or by cross polarization (CP) between 1H and ²⁹Si (recycling time of 5 s and contact time of 3 ms).

In silicon (²⁹Si) NMR, natural talc has a single peak at −97 ppm.

In silicon (²⁹Si) NMR, in contrast with natural talc, the spectrum of the synthetic phyllosilicate in accordance with the invention shows two peaks: one located at −95 ppm and the other located at −97 ppm, this being the case without the need for particle size fractionation to a size of less than 500 nm.

TGA (Thermogravimetric Analysis)

Method Used

The recordings were made using a Perkin Elmer Diamonds thermobalance.

For each analysis, about 20 mg of sample were required. During the analysis, the sample is subjected to a temperature increase ranging from 30° C. to 1200° C. with an increment of 10° C.·min⁻¹ under a stream of 100) mL·min⁻¹ of air.

The thermogravimetric analysis of a synthetic phyllosilicate in accordance with the invention shows lower thermal stability (at about 800° C.) than that of natural talc and it is characterized by four losses of mass, in contrast with natural talc which has only one, at about 900° C.

To establish these losses of mass, it is useful to refer to the article by Angela Dumas, François Martin, Christophe Le Roux, Pierre Micoud, Sabine Petit, Eric Ferrage, Jocelyne Brendle, Olivier Grauby and Mike Greenhill-Hooper: “Phyllosilicates synthesis: a way of accessing edges contributions in NMR and FTIR spectroscopies. Example of synthetic talc” (Phys. Chem. Minerals, published on Feb. 27, 2013).

X-Ray Diffraction

Method Used

Analysis of the X-ray diffractogram, especially with the aid of the materials and method used for X-ray diffraction analyses, is detailed in patent application FR 2 977 580.

Preferably, given that X-ray diffraction is performed only on solids, to visualize the absorption spectrum in a composition comprising at least an aqueous part, such as an emulsion, it is recommended to heat this composition to a temperature corresponding to a temperature greater than or equal to 100° C. (for example 120° C.) and less than or equal to 500° C. (for example 400° C.) so as to eliminate the adsorbed water part and, where appropriate, some or all of the organic compound(s) present in the composition.

The X-ray diffractogram of the synthetic phyllosilicate that is suitable for use in the invention has the same positions of the diffraction lines as those of natural talc, with the exception of one line. Specifically, natural talc has a diffraction line at 9.36 Å whereas the synthetic phyllosilicate in accordance with the invention has a diffraction line above 9.4 Å, which may be up to 9.8 Å.

More particularly, the synthetic phyllosilicate in accordance with the invention has a diffraction line greater than 9.4 Å and less than or equal to 9.8 Å.

The synthetic phyllosilicate in accordance with the invention preferably has a diffraction line greater than or equal to 9.5 Å, advantageously greater than or equal to 9.6 Å, and preferentially greater than or equal to 9.7 Å.

The synthetic phyllosilicate in accordance with the invention preferably has a diffraction line less than or equal to 9.7 Å, advantageously less than or equal to 9.6 Å, and preferentially less than or equal to 9.5 Å.

The synthetic phyllosilicate in accordance with the invention may also have a diffraction line between 4.60 Å and 4.80 Å, and/or a diffraction line between 3.10 Å and 3.20 Å and/or a diffraction line between 1.51 Å and 1.53 Å.

It should be noted that a synthetic phyllosilicate in accordance with the invention is free of interfoliar cations. Specifically, this characteristic is demonstrated by the absence of an X-ray diffraction line located at a distance of between 12.00 Å and 18.00 Å, usually revealing a swelling phase with interfoliar spaces in which are found interfoliar cations and possible water molecules.

A synthetic phyllosilicate that is suitable for use in the invention may be present in an amount ranging from 0.01% to 20% by weight, preferably ranging from 0.1% to 15% by weight, more preferentially ranging from 0.1% to 11% by weight, even more preferentially ranging from 0.5% to 11% by weight, better still ranging from 0.5% to 7% by weight, better still ranging from 1% to 6% by weight and even better still ranging from 2% to 5% by weight relative to the total weight of the composition.

It is understood that when a synthetic phyllosilicate in accordance with the invention is in gel form, the “% by weight” means the “% by weight of solids” or “% by weight of active material”.

The synthetic phyllosilicate that is suitable for use in the invention is in aqueous or aqueous-alcoholic gel form, and may constitute only part or alternatively all of the aqueous phase of the composition containing it.

According to a preferred implementation variant, a synthetic phyllosilicate that is suitable for use in the invention in aqueous or aqueous-alcoholic gel form is present in an amount ranging from 0.5% to 20% by weight of active material, preferably from 1% to 15% by weight and even more preferentially ranging from 2% to 10% by weight, relative to the total weight of the aqueous phase.

Electrolytes and Polyelectrolytes

Electrolytes

The term “electrolyte” means any molecule which has the capacity to dissociate, when it is dissolved in water or in any other ionizing medium, to give at least one ion. In other words, an electrolyte is a molecule comprising at least one ionizable group, for example a carboxylic acid.

As electrolytes that may be used in the composition according to the invention, mention may be made especially of mono-, di- or trivalent metal salts, and more particularly alkaline-earth metal salts and in particular barium, calcium and strontium salts, alkali metal salts, for example sodium and potassium salts, and also magnesium, beryllium, yttrium, lanthanum, cerium, praseodymium, neodymium, promethium, samarium, europium, gadolinium, terbium, dysprosium, holmium, erbium, thulium, ytterbium, lutetium, lithium, tin, zinc, silver, manganese, cobalt, nickel, iron, copper, rubidium, aluminum, silicon and selenium salts, and mixtures thereof.

The ions constituting these salts may be chosen, for example, from carbonates, bicarbonates, sulfates, phosphates, sulfonates, glycerophosphates, borates, bromides, chlorides, nitrates, acetates, hydroxides and persulfates and also ions of α-hydroxy acids (citrates, tartrates, lactates, malates) or of fruit acids, ions of β-hydroxy acids (salicylates, 2-hydroxyalkanoates, n-alkyl-salicylates and n-alkanoyl-salicylates), or alternatively ions of amino acids (aspartate, arginate, glycocholate, fumarate), for instance sodium (1-oxododecyl)-L-ethyl arginate hydrochloride.

Use may also be made of a mixture of these salts, including natural mixtures or mixtures whose composition is close to that of a natural mixture, and in particular of an aqueous mixture comprising from 30% to 35% of magnesium chloride, from 20% to 28% of potassium chloride, from 3% to 10% of sodium chloride, from 0.2% to 1% of calcium chloride, from 0.1% to 0.6% of magnesium bromide and from 0.1% to 0.5% of insoluble matter, said mixture being referred to herein as “Dead Sea Bath Salts” since it corresponds to the main salts contained in the Dead Sea.

Use may also be made of salts that are in the form of a solution or of a water containing them, and especially in the form of a spring or mineral water. In general, a mineral water is suitable for consumption, which is not always the case with a spring water. Each of these waters contains, inter alia, dissolved minerals and trace elements.

The spring water or mineral water used may be chosen, for example, from Vittel water, Vichy basin water, Uriage water, Roche-Posay water, Bourboule water, Enghien-les-Bains water, Saint Gervais-les-Bains water, Néris-les-Bains water, Allevard-les-Bains water, Digne water, Maizières water, Neyrac-les-Bains water, Lons-le-Saunier water, Eaux Bonnes water, Rochefort water, Saint Christau water, Fumades water, Avène water and Tercis-les-Bains water.

The electrolytes of the composition of the invention may also be chosen from the salts of active agents, for example salts derived from vitamins such as vitamin C (ascorbic acid) or vitamin A (retinol), or the salts of acidic active agents such as the salts of acidic filters or alternatively ammonium glycyrrhizinate or manganese gluconate. Examples of vitamin-based salts that may be mentioned include the ascorbyl phosphate of an alkali metal, alkaline-earth metal or transition metal such as magnesium, sodium, potassium, calcium or zinc; the retinyl phosphate of an alkali metal or alkaline-earth metal such as magnesium or potassium. Examples of filter-based salts that may be mentioned include salts of benzene-1,4-[bis(3-methylidenecamphor-10-sulfonic acid)], the sodium sulfonate of 2-hydroxy-4-methoxy-5-benzophenone (or benzophenone-5) acid, the disodium salt of 2,2′-dihydroxy-4,4′-dimethoxy-5,5′-disulfo-benzophenone (or benzophenone 9).

Among the electrolytes that are suitable for use in the invention, mention may also be made of the cucurbic acid derivatives corresponding to formula (I) below:

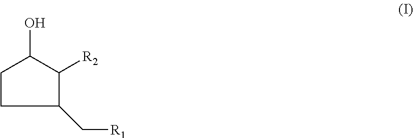

wherein:

R₁ represents a radical COOR₃, R₃ denoting a hydrogen atom or a C₁-C₄ alkyl radical, optionally substituted with one or more hydroxyl groups;

R₂ represents a saturated or unsaturated linear hydrocarbon-based radical containing from 1 to 18 carbon atoms or a saturated or unsaturated branched or cyclic hydrocarbon-based radical containing from 3 to 18 carbon atoms;

and also the optical isomers thereof, and corresponding salts.

Preferably, R₁ denotes a radical chosen from —COOH, —COOMe, —COO—CH₂—CH₃, —COO—CH₂—CH(OH)—CH₂OH, —COOCH₂—CH₂—CH₂OH and —COOCH₂—CH(OH)—CH₃. Preferentially, R₁ denotes a —COOH radical.

Preferentially, R₂ denotes a saturated or unsaturated linear hydrocarbon-based radical, preferably containing from 2 to 7 carbon atoms. In particular, R₂ may be a pentyl, pentenyl, hexyl or heptyl radical.

According to one embodiment, the compound of formula (I) is chosen from salts of 3-hydroxy-2-[(2Z)-2-pentenyl]cyclopentaneacetic acid or 3-hydroxy-2-pentylcyclopentaneacetic acid. Preferably, compound (I) is 3-hydroxy-2-pentylcyclopentaneacetic acid; this compound may especially be in the form of the sodium salt.

The salts of the compounds that may be used according to the invention are in particular chosen from salts of alkali metals, for example sodium or potassium; salts of alkaline-earth metals, for example calcium, magnesium or strontium; metal salts, for example zinc, aluminum, manganese or copper; ammonium salts of formula NH₄+; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts. Salts chosen from sodium, potassium, magnesium, strontium, copper, manganese and zinc salts are preferably used. The sodium salt is preferentially used.

The compound of formula (I) defined previously may be present in the composition according to the invention in a content ranging from 0.5% to 20% by weight, preferably from 1% to 15% by weight and more particularly from 1.5% to 10% by weight relative to the total weight of the composition.

Among the electrolytes according to the invention, mention may be made of the lipophilic salicylic acid derivatives in accordance with the invention corresponding to formula (II)

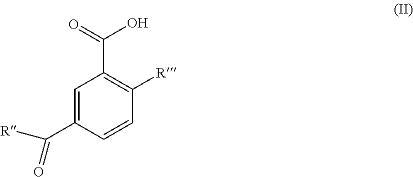

wherein:
the radical R″ denotes a linear, branched or cyclic saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from (a) halogen atoms, (b) the trifluoromethyl group, (c) hydroxyl groups in free form or esterified with an acid containing from 1 to 6 carbon atoms, or (d) a carboxyl function in free form or esterified with a lower alcohol containing from 1 to 6 carbon atoms;
R′″ is a hydroxyl group or an ester group of formula:

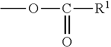

in which R¹ denotes a saturated or unsaturated, linear or branched aliphatic chain containing from 1 to 18 carbon atoms;
and also salts thereof derived from a mineral or organic base.

The lipophilic salicylic acid derivatives of formula (II) that may be used according to the invention are described in patents U.S. Pat. No. 6,159,479 and U.S. Pat. No. 5,558,871, FR 2 581 542, U.S. Pat. No. 4,767,750, EP 378 936, U.S. Pat. No. 5,267,407, U.S. Pat. No. 5,667,789, U.S. Pat. No. 5,580,549 and EP-A-570 230.

Preferentially, the radical R″ denotes a linear, branched or cyclic saturated aliphatic chain containing from 3 to 11 carbon atoms; an unsaturated chain containing from 3 to 17 carbon atoms and comprising one or more conjugated or unconjugated double bonds; said hydrocarbon-based chains possibly being substituted with one or more substituents, which may be identical or different, chosen from (a) halogen atoms, (b) the trifluoromethyl group, (c) hydroxyl groups in free form or esterified with an acid containing from 1 to 6 carbon atoms, or (d) a carboxyl function in free form or esterified with a lower alcohol containing from 1 to 6 carbon atoms;
R′″ is a hydroxyl group or an ester group of formula:

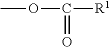

in which R¹ denotes a radical —O—(C═O)—(CH₂)n-CH₃ in which n is a number ranging from 0 to 14;
and also salts thereof obtained by salification with a mineral or organic base.

The compounds that are more particularly preferred are those in which the radical R″ is a C₃-C₁₁ alkyl group and R′″ denotes hydroxyl.

Other particularly advantageous compounds are those in which R represents a linoleic, linolenic or oleic acid-based chain.

Another group of particularly preferred compounds is formed by compounds in which the radical R″ denotes a C₃-C₁₁ alkyl group bearing a carboxyl function in free form or esterified with a lower alcohol containing from 1 to 6 carbon atoms and R′″ denotes hydroxyl.

The salts of the lipophilic salicylic acid derivatives of formula (II) may be obtained by salification with a mineral or organic base. Examples of mineral bases that may be mentioned include alkali metal hydroxides, for instance sodium hydroxide (lye) or potassium hydroxide (potash), alkaline-earth metal hydroxides or aqueous ammonia.

Among the organic bases, mention may be made of amines and alkanolamines. Quaternary salts, for instance those described in patent FR 2 607 498, are particularly advantageous.

The lipophilic salicylic acid derivatives of formula (II) that may be used according to the invention are described in patents U.S. Pat. No. 6,159,479 and U.S. Pat. No. 5,558,871, FR 2 581 542, FR 2 607 498, U.S. Pat. No. 4,767,750, EP 378 936, U.S. Pat. No. 5,267,407, U.S. Pat. No. 5,667,789, U.S. Pat. No. 5,580,549 and EP-A-570 230.

Among the compounds of formula (II) that are particularly preferred, mention may be made of:

5-n-octanoylsalicylic acid (or capryloylsalicylic acid); 5-n-decanoylsalicylic acid; 5-n-dodecanoyl-salicylic acid; 5-n-heptyloxysalicylic acid, and the corresponding salts thereof.

Use will be made more particularly of 5-n-octanoylsalicylic acid (or capryloylsalicylic acid) manufactured under the trade name Mexoryl SAB by the company Chimex (see page 139 of the International Cosmetic Ingredient Dictionary, 6^{th} Edition, Volume 1, published by the review Cosmetics, Toiletries and Fragrance Association, 1995).

In the compositions of the invention, the concentration of salicylic compound of formula (II) preferably ranges from 0.001% to 20% by weight, more preferentially from 0.01% to 15% by weight and even more preferentially from 0.05% to 5% by weight relative to the total weight of the composition.

Among the electrolytes that are suitable for use in the invention, mention may also be made of ionic, i.e. anionic or cationic, surfactants used in products for cleansing keratin materials and especially for facial and/or bodily skin.

The term “anionic surfactant” means a surfactant comprising, as ionic or ionizable groups, only anionic groups. These anionic groups are preferably chosen from CO₂H, CO₂⁻, SO₃H, SO₃⁻, OSO₃H, OSO₃⁻, —H₂PO₃, —HPO₃⁻, —PO₃²⁻, —H₂PO₂, ═HPO₂, —HPO₂⁻, ═PO₂⁻, ═POH and ═PO⁻ groups. The anionic surfactants may be oxyalkylenated and then preferably comprise from 1 to 50 ethylene oxide units and better still from 1 to 10 ethylene oxide units.

Preferably, the anionic surfactants are chosen from isethionate, sulfate, sulfonate or carboxylate anionic surfactants, preferentially from sulfonate anionic surfactants and carboxylate anionic surfactants.

According to the invention, included in the sulfonate anionic surfactants are sulfonate anionic surfactants without a carboxylate group; and included in the carboxylate anionic surfactants are carboxylate anionic surfactants which can optionally also comprise a sulfate or sulfonate group, for example.

The sulfate or sulfonate anionic surfactants without a carboxylic group that can be used in the composition according to the invention as anionic surfactants can be chosen in particular from alkyl sulfates, alkyl ether sulfates, alkylamido ether sulfates, alkylaryl polyether sulfates, monoglyceride sulfates, alkylsulfonates, alkylamidesulfonates, alkylarylsulfonates, α-olefin sulfonates, paraffin sulfonates, N-acyltaurates, N-methyl-N-acyltaurates, and the corresponding acid forms, the alkyl and acyl groups of all these compounds preferably comprising from 6 to 30 carbon atoms, better still from 12 to 24, or even from 16 to 22, carbon atoms, and the aryl group preferably denoting a phenyl or benzyl group.

The carboxylate anionic surfactants that may be used in the composition according to the invention as anionic surfactants can be chosen from alkyl sulfosuccinates, alkyl ether sulfosuccinates, alkylamide sulfosuccinates, acyl glycinates, acylsarcosinates and acyl glutamates, and the corresponding acid forms, the alkyl and/or acyl groups of these compounds comprising from 6 to 30 carbon atoms, better still from 12 to 24, or even from 16 to 22, carbon atoms.

Use may also be made of alkyl monoesters of polyglycoside-polycarboxylic acids such as alkyl polyglycoside citrates, alkyl polyglycoside tartrates and alkyl polyglycoside sulfosuccinates, and alkylsulfosuccinamates, the alkyl or acyl group of these compounds comprising from 6 to 30 carbon atoms, better still from 12 to 24, or even from 16 to 22, carbon atoms; use may also be made of the salts thereof.

Use may also be made of acyllactylates, the acyl group of which comprises from 6 to 30 carbon atoms, better still from 8 to 20 carbon atoms, or even from 12 to 24 carbon atoms. Mention may also be made of alkyl-D-galactosideuronic acids, polyoxyalkylenated (C₁₄-C₃₀)alkyl ether carboxylic acids, polyoxyalkylenated (C₁₄-C₃₀)alkyl(C₆-C₃₀)aryl ether carboxylic acids, polyoxyalkylenated (C₁₄-C₃₀)alkylamido ether carboxylic acids; and also the salts of all these compounds; the compounds preferably comprising from 2 to 50 ethylene oxide units; and also mixtures thereof.

Preferably, the composition comprises one or more anionic surfactants chosen from:
C₆-C₂₄ and especially C₁₂-C₂₀ alkyl sulfates,
C₆-C₂₄ and in particular C₁₂-C₂₀ alkyl ether sulfates; preferably comprising from 2 to 20 ethylene oxide units,
C₆-C₂₄ and especially C₁₂-C₂₀ acylglutamates; especially stearoylglutamates,
C₆-C₂₄ and especially C₁₂-C₂₀ acylsarcosinates; especially palmitoylsarcosinates,
C₆-C₂₄ and especially C₁₂-C₂₀ acyllactylates; especially behenoyl-lactylates,
(C₆-C₂₄)alkyl ether carboxylates, preferably (C₁₂-C₂₀)alkyl ether carboxylates,
C₆-C₂₄ and especially C₁₂-C₂₀ alkylsulfosuccinates; especially laurylsulfosuccinates,
acylisethionates such as cocoyl isethionates and/or lauroyl isethionates.

The salified forms are in particular alkali metal salts such as sodium salts, ammonium salts, amine salts, amino alcohol salts or alkaline-earth metal salts, for example magnesium salts. Alkali metal or alkaline-earth metal salts and in particular the sodium or magnesium salts are preferably used.

According to one embodiment, the composition according to the invention may comprise at least one cationic surfactant. The cationic surfactants that may be used according to the present invention are especially salts of optionally polyoxyalkylenated primary, secondary or tertiary fatty amine salts, quaternary ammonium salts, imidazoline derivatives and amine oxides of cationic nature, and mixtures thereof.

Examples of quaternary ammonium salts include:
those that have the general formula (III) below:

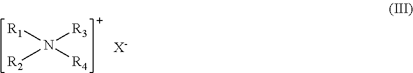

in which the radicals R₁ to R₄, which may be identical or different, represent a linear or branched aliphatic radical containing from 1 to 30 carbon atoms, or an aromatic radical such as aryl or alkylaryl. The aliphatic radicals may comprise heteroatoms especially such as oxygen, nitrogen, sulfur and halogens. The aliphatic radicals are chosen, for example, from alkyl, alkoxy, polyoxy(C₂-C₆)alkylene, alkylamide, (C₁₂-C₂₂)-alkylamido(C₂-C₆)alkyl, (C₂-C₂₂)alkylacetate and hydroxyalkyl radicals, comprising from about 1 to 30 carbon atoms. X is an anion chosen from the group of halides, phosphates, acetates, lactates, (C₂-C₆)alkyl sulfates and alkyl or alkylaryl sulfonates. Preferably, R₁ and R₂ denote a C₁-C₄ alkyl or a C₁-C₄ hydroxyalkyl.
quaternary ammonium salts of imidazolinium, for instance the salt of formula (IV) below:

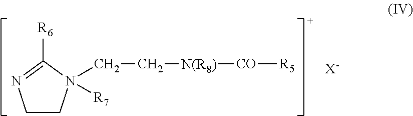

in which R₅ represents an alkenyl or alkyl radical comprising from 8 to 30 carbon atoms, for example copra fatty acid derivatives, R₆ represents a hydrogen atom, a C₁-C₄ alkyl radical or an alkenyl or alkyl radical containing from 8 to 30 carbon atoms. R₇ represents a C₁-C₄ alkyl radical, R₈ represents a hydrogen atom or a C₁-C₄ alkyl radical, X− is an anion chosen from the group of halides, phosphates, acetates, lactates, alkyl sulfates and alkyl or alkylaryl sulfonates. Preferably, R₅ and R₆ denote a mixture of alkenyl or alkyl radicals comprising from 12 to 21 carbon atoms, for example derived from tallow fatty acids, R₇ denotes a methyl group and R₈ denotes a hydrogen atom.
the quaternary diammonium salts of formula (V):

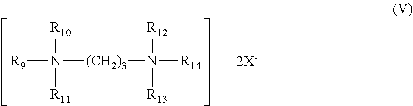

in which R₉ denotes an aliphatic radical comprising about from 16 to 30 carbon atoms. R₁₀, R₁₁, R₁₂, R₁₃ and R₁₄, which may be identical or different, are chosen from hydrogen and an alkyl radical comprising from 1 to 4 carbon atoms, and X is an anion chosen from the group of halides, acetates, phosphates, nitrates and methyl sulfates.
quaternary ammonium salts comprising at least one ester function, for example those of formula (VI) below:

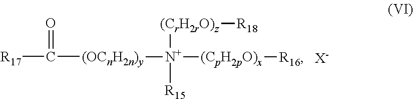

wherein:
R₁ is chosen from C₁-C₆ alkyl radicals and C₁-C₆ hydroxyalkyl or dihydroxyalkyl radicals;
R₁₆ is chosen from:
   the radical

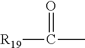

linear or branched, saturated or unsaturated C₁-C₂₂ hydrocarbon-based radicals R₂₀,
a hydrogen atom,
R₁₈ is chosen from:
   the radical

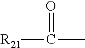

linear or branched, saturated or unsaturated C₁-C₆ hydrocarbon-based radicals R₂₂,
a hydrogen atom,
R₁₇, R₁₉ and R₂₁, which may be identical or different, are chosen from linear or branched, saturated or unsaturated C₇-C₂₁ hydrocarbon-based radicals;
n, p and r, which may be identical or different, are integers ranging from 2 to 6;
y is an integer ranging from 1 to 10;
x and z, which may be identical or different, are integers ranging from 0 to 10;
X⁻ is a simple or complex, organic or mineral anion;

with the proviso that the sum x+y+z is from 1 to 15, that when x is 0 then R₁₆ denotes R₂₀, and that when z is 0 then R₁₈ denotes R₂₂.

The alkyl radicals R₁₅ may be linear or branched, and more particularly linear.

Preferably, R₁₅ denotes a methyl, ethyl, hydroxyethyl or dihydroxypropyl radical and more particularly a methyl or ethyl radical.

Advantageously, the sum x+y+z is from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it preferably contains 1 to 3 carbon atoms.

Advantageously, R₁₇, R₁₉ and R₂₁, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are equal to 0 or 1.

Advantageously, y is equal to 1.

n, p and r, which may be identical or different, are preferably 2 or 3 and even more particularly are equal to 2.

The anion is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion that is compatible with the ammonium bearing an ester function.

The anion X⁻ is even more particularly chloride or methyl sulfate.

Use is made more particularly of the ammonium salts of formula (VII) in which:
R₁₅ denotes a methyl or ethyl radical;
x and y are equal to 1;
z is equal to 0 or 1;
n, p and r are equal to 2;
R₁₆ is chosen from:
   the radical

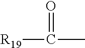

methyl, ethyl or C₁₄-C₂₂ hydrocarbon-based radicals,
a hydrogen atom;
R₁₈ is chosen from:
   the radical

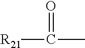

a hydrogen atom;

R₁₇, R₁₉ and R₂₁, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

Advantageously, the hydrocarbon-based radicals are linear.

Among the quaternary ammonium salts of formula (III), preference is given, on the one hand, to tetraalkylammonium chlorides, for instance dialkyldimethylammonium or alkyltrimethylammonium chlorides in which the alkyl radical comprises approximately 12 to 22 carbon atoms, more particularly behenyltrimethylammonium chloride, distearyldimethylammonium chloride, cetyltrimethylammonium chloride and benzyldimethylstearylammonium chloride, or else, on the other hand, to palmitylamidopropyltrimethylammonium chloride or the stearamidopropyldimethyl(myristyl acetate)ammonium chloride sold in particular under the name Ceraphyl 70 by the company Van Dyk.

Examples of compounds of formula (VI) that may be mentioned include the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular), and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart by the company Cognis, Stepanquat by the company Stepan, Noxamium by the company Ceca, and Rewoquat WE 18 and Rewoquat W75 by the company Degussa.

Use may also be made of the ammonium salts containing at least one ester function that are described in patents U.S. Pat. No. 4,874,554 and U.S. Pat. No. 4,137,180.

Quaternary diammonium salts of formula (VI) that are suitable for use in the invention comprise, in particular, propanetallowdiammonium chloride.

According to a preferred embodiment, the electrolyte that is suitable for use in the invention is chosen from an aluminum and/or zirconium salt or complex, a silver salt such as silver chloride, a zinc salt, and mixtures thereof, preferably chosen from an aluminum and/or zirconium salt or complex, and even more particularly aluminum chlorohydrate.

Among the zinc salts, mention may be made of zinc pyrrolidonecarboxylate (more commonly known as zinc pidolate), zinc sulfate, zinc chloride, zinc lactate, zinc gluconate and zinc phenolsulfonate.

According to one embodiment, among the aluminum salts or complexes, mention may be made of aluminum potassium double sulfate, a deodorant active agent of chemical formula KAl(SO₄)₂.12H₂O, also known as potassium alum.

According to a preferred embodiment, the electrolyte that is suitable for use in the invention is chosen from aluminum and/or zirconium antiperspirant salts or complexes. They are preferably chosen from aluminum halohydrates; aluminum zirconium halohydrates, complexes of zirconium hydroxychloride and of aluminum hydroxychloride with or without an amino acid, such as those described in U.S. Pat. No. 3,792,068, and mixtures thereof.

The term “antiperspirant active agent” means a salt which, by itself, has the effect of reducing the flow of sweat, of reducing the sensation on the skin of moisture associated with human sweat or of masking human sweat.

Among the aluminum salts, mention may in particular be made of aluminum chlorohydrate in activated or unactivated form, aluminum chlorohydrex, the aluminum chlorohydrex-polyethylene glycol complex, the aluminum chlorohydrex-propylene glycol complex, aluminum dichlorohydrate, the aluminum dichlorohydrex-polyethylene glycol complex, the aluminum dichlorohydrex-propylene glycol complex, aluminum sesquichlorohydrate, the aluminum sesquichlorohydrex-polyethylene glycol complex, the aluminum sesquichlorohydrex-propylene glycol complex, aluminum sulfate buffered with sodium aluminum lactate, and mixtures thereof.

Among the aluminum zirconium salts, mention may be made in particular of aluminum zirconium octachlorohydrate, aluminum zirconium pentachlorohydrate, aluminum zirconium tetrachlorohydrate and aluminum zirconium trichlorohydrate, and mixtures thereof.

The complexes of zirconium hydroxychloride and of aluminum hydroxychloride with an amino acid are generally known under the name ZAG (when the amino acid is glycine). Among these products, mention may be made of the aluminum zirconium octachlorohydrex-glycine complex, the aluminum zirconium pentachlorohydrex-glycine complex, the aluminum zirconium tetrachlorohydrex-glycine complex and the aluminum zirconium trichlorohydrex-glycine complex, and mixtures thereof.

Aluminum sesquichlorohydrate is sold especially under the trade name Reach 301® by the company Summitreheis.

Among the aluminum and zirconium salts, mention may be made of the complexes of zirconium hydroxychloride and of aluminum hydroxychloride with an amino acid such as glycine, having the INCI name: Aluminum Zirconium Tetrachlorohydrex Gly, for example the product sold under the name Reach AZP-908-SUF® by the company Summitreheis.

Use will be made more particularly of aluminum chlorohydrate in activated or unactivated form sold especially under the trade names Locron S FLA®, Locron P® and Locron L.ZA® by the company Clariant; under the trade names Microdry Aluminum Chlorohydrate®, Micro-Dry 323®, Chlorhydrol® 50, Reach® 103 and Reach® 501 by the company Summitreheis; under the trade name Westchlor 200® by the company Westwood; under the trade name Aloxicoll PF 40® by the company Guilini Chemie; Cluron 50%® by the company Industria Quimica Del Centro; or Clorohidroxido Aluminio SO A 50%® by the company Finquimica.

Thus, according to a preferred embodiment, the aluminum salt used in a composition according to the invention is a chlorohydrate.

For example, the electrolyte(s) may be present in the composition according to the invention in an amount ranging from 0.1% to 30% by weight of active material, preferentially ranging from 0.1% to 20% by weight, even more preferentially ranging from 0.2% to 15% by weight, better still ranging from 0.3% to 10% by weight and even better still ranging from 0.5% to 5% by weight relative to the total weight of the composition. In one embodiment of the invention, for example when the electrolyte is chosen from an aluminum and/or zirconium salt, an aluminum and/or zirconium complex, and mixtures thereof, the electrolyte(s) may be present in the composition according to the invention in an amount ranging from 1% to 30% by weight, preferably ranging from 2% to 30% by weight, even more preferentially ranging from 10% to 30% by weight and better still from 15% to 30% by weight relative to the total weight of the composition.

This amount of electrolyte(s) varies especially according to the electrolyte used and the desired aim for the final composition.

Polyelectrolytes

The term “polyelectrolyte” means a macromolecular substance which has the capacity to dissociate, when it is dissolved in water or in any other ionizing medium, to give at least one ion. In other words, a polyelectrolyte is a polymer comprising at least one ionizable monomer.

In particular, the polyelectrolyte may give polyions, for example polyanions, when it is dissociated in water. A polyelectrolyte may be a polyacid (depending on the definition of the electrolytes above), a polybase or a polysalt. In the context of the invention, it is preferably a polysalt.

Preferably, the polyelectrolyte included in the cosmetic compositions according to the present invention is a branched and/or crosslinked anionic polymer.

Advantageously, it also differs from the polymer in aqueous dispersion.

The counterions of the polyions formed during the dissociation may be of any nature, mineral or organic.

In particular, when the polyelectrolyte is a branched or crosslinked anionic polymer, the cations may be alkali metal or alkaline-earth metal cations such as sodium or potassium, or alternatively the ammonium ion.

The sodium cation Na+ is preferred, which is why it is mainly mentioned in the list of polyelectrolytes that follows, without this constituting any limitation to this specific counterion.

Polyelectrolytes that may be mentioned include:
anionic polysaccharides including polysaccharide sulfates; xanthans; carrageenans; alginate-based compounds; crosslinked or non-crosslinked hyaluronic acids (HAs) and salts thereof; sodium carboxymethylcellulose and all ionizable derivatives of cellulose; starch-based grafted copolymers such as the Waterlock® products (A-180 and G-400, for example) from Grain Processing Corporation, crosslinked sodium starch glycolate in powder form;
cationic polymers including chitosans and polymeric quaternary ammonium salts;
crosslinked and/or neutralized 2-methyl-2-[(1-oxo-2-propenyl)amino]-1-propanesulfonic acid (AMPS®) homopolymers and copolymers;
acrylic homopolymers and copolymers such as sodium polyacrylates such as Norsocryl® S35 sold especially by the company Atofina, or Cosmedia® SP sold especially by the company Cognis;
polyacrylic acids of the type such as Synthalen K®;
polyacrylic acid/alkyl acrylate copolymers of the type such as Pemulen®;
and mixtures thereof.

Sodium polyacrylate and the acrylamide/AMPS copolymer, and copolymers thereof, are most particularly suitable for use in the invention.

Polysaccharide Sulfates

The polysaccharide sulfates used according to the invention are chosen from dextran sulfate and the polysaccharide sulfates derived from the red alga Porphyridium sp.

Advantageously, the degree of sulfation of the polysaccharides may range from 1% to 30% by weight, relative to the weight of the polysaccharide. Preferably, this degree of sulfation may range from 2% to 25% by weight.

The polysaccharide may optionally be acetylated. The degree of acetylation may range from 0 to 10% by weight (weight content of acetyl units relative to the total weight of the polymer).

The polysaccharide used according to the invention preferably has a weight-average molecular weight of between 1000 and 20×106 daltons, preferentially between 2000 and 10×106 Da, preferentially between 5000 and 5×106 Da and more preferentially between 10 000 and 1×106 Da.

The polysaccharide sulfate used according to the invention may be dextran sulfate. The dextran sulfate sold under the trade name Dextran Sulfate 10 Sodium Salt CG by the company PK Chemicals may be used.

The polysaccharide sulfate used according to the invention may be chosen from polysaccharide sulfates derived from the red alga Porphyridium sp., such as the product sold under the name Alguard®® by the company Frutarom. Patent application EP-A-311496 especially describes a process for preparing such polysaccharides. These polysaccharide sulfates contain glucuronic acid, xylose, glucose and galactose which are sulfated.

Xanthans

Xanthan is a heteropolysaccharide produced on an industrial scale by the aerobic fermentation of the bacterium Xanthomonas campestris. Its structure consists of a main chain of β(1,4)-linked β-D-glucoses, similar to cellulose. One glucose molecule in two bears a trisaccharide side chain composed of an α-D-mannose, a β-D-glucuronic acid and a terminal β-D-mannose. The internal mannose residue is generally acetylated on carbon 6. About 30% of the terminal mannose residues bear a pyruvate group linked in chelated form between carbons 4 and 6. The charged pyruvic acids and glucuronic acids are ionizable, and are thus responsible for the anionic nature of xanthan (negative charge down to a pH equal to 1). The content of the pyruvate and acetate residues varies according to the bacterial strain, the fermentation process, the conditions after fermentation and the purification steps. These groups may be neutralized in commercial products with Na⁺, K⁺ or Ca²⁺ ions (Satia company, 1986). The neutralized form may be converted into the acid form by ion exchange or by dialysis of an acidic solution.

Xanthan gums have a molecular weight of between 1 000 000 and 50 000 000 and a viscosity of between 0.6 and 1.65 Pa·s for an aqueous composition containing 1% of xanthan gum (measured at 25° C. on a Brookfield viscometer of LVT type at 60 rpm).

Xanthan gums are represented, for example, by the products sold under the names Rhodicare by the company Rhodia Chimie, under the name Satiaxane™ by the company Cargill Texturizing Solutions (for the food, cosmetic and pharmaceutical industries), under the name Novaxan™ by the company ADM, and under the names Kelzan® and Keltrol® by the company CP-Kelco.

Carrageenans

Carrageenans are anionic polysaccharides constituting the cell walls of various red algae (Rhodophyceae) belonging to the Gigartinacae, Hypneaceae, Furcellariaceae and Polyideaceae families. They are generally obtained by hot aqueous extraction from natural strains of said algae. These linear polymers, formed by disaccharide units, are composed of two D-galactopyranose units linked alternately by α(1,3) and β(1,4) bonds. They are highly sulfated polysaccharides (20%-50%) and the α-D-galactopyranosyl residues may be in 3,6-anhydro form. Depending on the number and position of sulfate-ester groups on the repeating disaccharide of the molecule, several types of carrageenans are distinguished, namely: kappa-carrageenans, which bear one sulfate-ester group, iota-carrageenans, which bear two sulfate-ester groups, and lambda-carrageenans, which bear three sulfate-ester groups.

Carrageenans are composed essentially of potassium, sodium, magnesium, triethanolamine and/or calcium salts of polysaccharide sulfate esters.

Carrageenans are sold especially by the company SEPPIC under the name Solagum®, by the company Gelymar under the names Carragel®, Carralact® and Carrasol®, by the company Cargill under the names Satiagel™ and Satiagum™, and by the company CP-Kelco under the names Genulacta®, Genugel® and Genuvisco®.

Alginate-Based Compound

For the purposes of the invention, the term “alginate-based compound” means alginic acid, alginic acid derivatives and salts of alginic acid (alginates) or of said derivatives.

Preferably, the alginate-based compound is water-soluble.

Alginic acid, a natural substance resulting from brown algae or certain bacteria, is a polyuronic acid composed of 2 uronic acids linked by 1,4-glycosidic bonds: β-D-manuronic (M) acid and α-L-glucuronic (G) acid.

Alginic acid is capable of forming water-soluble salts (alginates) with alkali metals such as sodium, potassium or lithium, substituted cations of lower amine and of ammonium such as methylamine, ethanolamine, diethanolamine or triethanolamine. These alginates are water-soluble in aqueous medium at a pH equal to 4, but dissociate into alginic acid at a pH below 4.

This (these) alginate-based compound(s) are capable of crosslinking in the presence of at least one crosslinking agent, by formation of ionic bonds between said alginate-based compound(s) and said crosslinking agent(s). The formation of multiple crosslinks between several molecules of said alginate-based compound(s) leads to the formation of a water-insoluble gel.

Use is preferably made of alginate-based compounds with a weight-average molecular mass ranging from 10 000 to 1 000 000, preferably from 15 000 to 500 000 and better still from 20 000 to 250 000.

According to a preferred embodiment, the alginate-based compound is alginic acid and/or a salt thereof.

Advantageously, the alginate-based compound is an alginate salt, and preferably sodium alginate.

The alginate-based compound may be chemically modified, especially with urea or urethane groups or by hydrolysis, oxidation, esterification, etherification, sulfatation, phosphatation, amination, amidation or alkylation reaction, or by several of these modifications.

The derivatives obtained may be anionic, cationic, amphoteric or nonionic.

The alginate-based compounds that are suitable for use in the invention may be represented, for example, by the products sold under the names Kelcosol, Satialgine™, Cecalgum™ or Algogel™ by the company Cargill Products, under the name Protanal™ by the company FMC Biopolymer, under the name Grindsted® Alginate by the company Danisco, under the name Kimica Algin by the company Kimica, and under the names Manucol® and Manugel® by the company ISP.

Polymeric Quaternary Ammonium Salts
Polymeric quaternary ammonium salts are cationic or amphoteric polymers containing at least one quaternized nitrogen atom. Polymeric quaternary ammonium salts that may especially be mentioned include the Polyquaternium products (CTFA name), which afford softness and creaminess to the foaming cream. These polymers may preferably be chosen from the following polymers:
Polyquaternium 5, such as the product Merquat 5 sold by the company Nalco;
Polyquaternium 6, such as the product Salcare SC 30 sold by the company Ciba, and the product Merquat 100 sold by the company Nalco;
Polyquaternium 7, such as the products Merquat S, Merquat 2200 and Merquat 550 sold by the company Nalco, and the product Salcare SC 10 sold by the company Ciba;
Polyquaternium 10, such as the product Polymer JR400 sold by the company Amerchol;
Polyquaternium 11, such as the products Gafquat 755, Gafquat 755N and Gafquat 734 sold by the company ISP;
Polyquaternium 15, such as the product Rohagit KF 720 F sold by the company Röhm;
Polyquaternium 16, such as the products Luviquat FC905, Luviquat FC370, Luviquat HM552 and Luviquat FC550 sold by the company BASF;
Polyquaternium 22, such as the product Merquat 280 sold by the company Nalco;
Polyquaternium 28, such as the product Styleze CC10 sold by the company ISP;
Polyquaternium 39, such as the products Merquat Plus 3330 and Merquat 3330PR sold by the company Nalco;
Polyquaternium 44, such as the product Luviquat Care sold by the company BASF;
Polyquaternium 46, such as the product Luviquat Hold sold by the company BASF;
Polyquaternium 47, such as the product Merquat 2001 sold by the company Nalco.
Preferably, the quaternary ammonium salts are chosen from Polyquaternium-7, Polyquaternium-10, Polyquaternium-39 and Polyquaternium-47, and mixtures thereof.

Crosslinked and/or Neutralized 2-Acrylamido-2-Methylpropanesulfonic Acid Polymers and Copolymers

The polymers used that are suitable as aqueous gelling agent for the invention may be crosslinked or non-crosslinked homopolymers or copolymers comprising at least the 2-acrylamidomethylpropanesulfonic acid (AMPS®) monomer, in a form partially or totally neutralized with a mineral base other than aqueous ammonia, such as sodium hydroxide or potassium hydroxide.

They are preferably totally or almost totally neutralized, i.e. at least 90% neutralized.

These AMPS® polymers according to the invention may be crosslinked or non-crosslinked.

As water-soluble or water-dispersible AMPS homopolymers suitable for use in the invention, mention may be made, for example, of crosslinked or non-crosslinked polymers of sodium acrylamido-2-methylpropanesulfonate, such as that used in the commercial product Simulgel 800 (CTFA name: Sodium Polyacryloyldimethyl Taurate), crosslinked ammonium acrylamido-2-methylpropanesulfonate polymers (INCI name: Ammonium Polyacryldimethyltauramide) such as those described in patent EP 0 815 928 B1 and such as the product sold under the trade name Hostacerin AMPS® by the company Clariant.

As preferred water-soluble or water-dispersible AMPS homopolymers in accordance with the invention, mention may be made of crosslinked ammonium acrylamido-2-methylpropanesulfonate polymers.

As water-soluble or water-dispersible AMPS copolymers in accordance with the invention, examples that may be mentioned include:
crosslinked acrylamide/sodium acrylamido-2-methylpropanesulfonate copolymers, such as that used in the commercial product Sepigel 305 (CTFA name: Polyacrylamide/C₃-C₁₄ Isoparaffin/Laureth-7) or that used in the commercial product sold under the name Simulgel 600 (CTFA name: Acrylamide/Sodium acryloyldimethyltaurate/Isohexadecane/Polysorbate-80) by the company SEPPIC;
copolymers of AMPS® and of vinylpyrrolidone or vinylformamide, such as that used in the commercial product sold under the name Aristoflex AVC® by the company Clariant (CTFA name: Ammonium Acryloyldimethyltaurate/VP copolymer) but neutralized with sodium hydroxide or potassium hydroxide;
copolymers of AMPS® and of sodium acrylate, for instance the AMPS/sodium acrylate copolymer, such as that used in the commercial product sold under the name Simulgel EG® by the company SEPPIC or under the trade name Sepinov EM (CTFA name: Hydroxyethyl acrylate/Sodium acryloyldimethyltaurate copolymer);
copolymers of AMPS® and of hydroxyethyl acrylate, for instance the AMPS®/hydroxyethyl acrylate copolymer, such as that used in the commercial product sold under the name Simulgel NS® by the company SEPPIC (CTFA name: Hydroxyethyl acrylate/sodium acryloyldimethyltaurate copolymer (and) squalane (and) polysorbate 60), or such as the product sold under the name sodium acrylamido-2-methylpropanesulfonate/hydroxyethyl acrylate copolymer, such as the commercial product Sepinov EMT 10 (INCI name: Hydroxyethyl acrylate/Sodium acryloyldimethyltaurate copolymer).

According to a preferred embodiment, the polyelectrolyte is chosen from a crosslinked hyaluronic acid, a non-crosslinked hyaluronic acid, salts thereof and mixtures thereof. The salts may be salts of monovalent cations such as those of alkali metals (lithium, sodium, potassium, etc.) or salts of divalent cations such as those of alkaline-earth metals (beryllium, magnesium, calcium, strontium, etc.).

Hyaluronic acid is a linear, non-sulfated glycosaminoglycan composed of D-glucuronic acid and N-acetyl-D-glucosamine repeating units.

According to the invention, the hyaluronic acid derivative preferably has a number-average molecular weight ranging from 500 Da to 10 MDa and more particularly ranging from 2 KDa to 2 MDa.

As hyaluronic acids that are suitable for use in the present invention, mention may be made especially of hyaluronic acids of animal origin, or obtained via biotechnology. They are linear or crosslinked, such as those sold under the name Hylaform® by the company Genzyme, or hyaluronic acids of genetic origin, including those intended for periorbital or peribuccal surface wrinkles, such as those sold under the name Restylane Fine Lines® by Laboratoire Q-Med, or intended for deep wrinkles or labio-mandibular and oval depressions of the face, such as those sold under the names Perlane® and Restylane Sub-Q® by Laboratoire Q-Med.

Preferably, as hyaluronic acids that are suitable for use in the present invention, mention may be made of those sold under the name Restylane® by Laboratoire Q-Med and under the name Surgiderm® by Laboratoire Corneal.

The following forms of sodium hyaluronate may also be advantageously considered in the context of the invention:
of molecular weight between 20 and 50 kg/mol sold by the company Soliance under the name Renovyal LO®,
of molecular weight between 100 and 300 kg/mol sold by the company Soliance under the name Primalhyal 300®,
of molecular weight between 500 and 1200 kg/mol sold by the company Soliance under the name Cristalhyal LO®,
of molecular weight between 10 and 200 kg/mol sold by the company Bioland under the name Bioha®,
of molecular weight between 30 and 60 kg/mol sold by the company Evonik Goldschmidt under the name Hyacare 50®,
of molecular weight between 100 and 140 kg/mol sold by the company Contripro under the name Hyactive 120®.

Among the hyaluronic acid salts, mention may be made especially of the sodium salts, the potassium salts, the zinc salts and the silver salts, and mixtures thereof.

More particularly, as hyaluronic acid salts, mention may be made of potassium hyaluronate and sodium hyaluronate, preferably sodium hyaluronate.

It is understood that the amount of polyelectrolyte(s) is liable to vary significantly depending on the nature of the polyelectrolyte and the intended aim of the composition containing it.

For example, the polyelectrolyte(s) may be present in the composition according to the invention in an amount ranging from 0.1% to 30% by weight of active material, preferentially ranging from 0.1% to 20% by weight, even more preferentially ranging from 0.2% to 15% by weight, better still ranging from 0.3% to 10% by weight and even better still ranging from 0.5% to 5% by weight relative to the total weight of the composition.

The electrolyte(s) and/or the polyelectrolyte(s) may be present in the composition according to the invention in a total amount ranging from 0.1% to 30% by weight of active material, preferentially ranging from 0.1% to 20% by weight, even more preferentially ranging from 0.2% to 15% by weight, better still ranging from 0.3% to 10% by weight and even better still ranging from 0.5% to 5% by weight relative to the total weight of the composition.

Film-Forming Polymers

As mentioned above, when the electrolyte is chosen from an aluminum and/or zirconium salt, an aluminum and/or zirconium complex, and mixtures thereof, preferably chosen from aluminum halohydrates, aluminum zirconium halohydrates, complexes of zirconium hydroxychloride and of aluminum hydroxychloride, with or without an amino acid, and mixtures thereof, a composition according to the invention may also comprise (c) at least one film-forming polymer chosen from a hydrophilic film-forming polymer and a hydrophobic film-forming polymer, and mixtures thereof.

Hydrophobic Film-Forming Polymers

The term “hydrophobic film-forming polymer” or “oily film-forming polymer” means any polymer:
(1) which is capable of forming, by itself or in the presence of an auxiliary film-forming agent, a continuous or discontinuous film that adheres to a support, especially to the skin; and
(2) for which the film formed is capable of absorbing an amount of water of less than or equal to 30% by weight relative to the weight of the dry polymer film (before immersion in water) when it is dipped into liquid water.

The amount of water absorbed by the hydrophobic polymers according to the present invention may be measured under the following conditions:

To measure the amount of water absorbed, also known as the water uptake, 12 g of an aqueous or aqueous-alcoholic solution comprising 7% by weight of polymer are poured into an aluminum crucible 5.5 cm in diameter, to form a film. The inner surface of the aluminum crucible is covered with a Teflon support disk so as to limit the undesirable edge effects and to facilitate the shrinkage of the film. The system is left to evaporate for 24 hours with ventilation, so as to allow optimum drying. A circular film measuring between 300 and 350 μm in thickness is obtained, which is removed from the aluminum crucible. The film is then cut into two rectangles of 1×2 cm.

One of the rectangular films obtained is weighed when dry, which corresponds to the mass of the film before immersion in the water, or the mass of the dry film. The same film is then dipped into a 30 mL flask filled with water, for a duration of 60 minutes.

After each immersion in water, the excess surface water is removed by very gently pressing the film onto cotton paper and the film is weighed, which corresponds to the mass of the film after immersion in water. The percentage of water absorbed or the water uptake of the polymer after 60 minutes is calculated according to the following equation:

% water absorbed=(m_{Film after immersion}−m_{Dry film})/m_{Dry film}

The operation is repeated three times for each of the polymers tested. The average of the three percentages of absorption is calculated, to deduce therefrom the percentage of water absorbed by the polymer.

Needless to say, a person skilled in the art will choose the polymers as a function of their compatibility with the compound chosen from an aluminum and/or zirconium salt, an aluminum and/or zirconium complex, and mixtures thereof used in a composition according to the invention.

According to a particular form of the invention, the hydrophobic film-forming polymer(s) are synthetic polymers.

The term “synthetic polymer” means any polymer obtained chemically or via production in an organism of the elements necessary for this production.

The synthetic hydrophobic polymers used according to the invention may preferably comprise:

(i) polymers of interpenetrating polymer network (IPN) type;

(ii) grafted silicone polymers;

(iii) non-neutralized (meth)acrylic acid/N-tert-butylacrylamide copolymers;

(iv) non-neutralized crotonic acid/vinyl acetate copolymers;

(v) tetrapolymers of (meth)acrylic acid, of (meth)acrylates and of C₈-C₂₄ alkyl (meth)acrylate;

(vi) film-forming pseudoblocks:

(vii) hydrocarbon-based block copolymers; and

(viii) mixtures thereof.

Interpenetrating Polymer Network

According to a first variant, the hydrophobic film-forming polymers are polymers of interpenetrating polymer network type.

For the purposes of the present invention, the expression “interpenetrating polymer network” means a blend of two interlaced polymers, obtained by simultaneous polymerization and/or crosslinking of two types of monomer, the blend obtained having a single glass transition temperature range.

An IPN that is particularly preferred is in the form of an aqueous dispersion of particles with a number-average size ranging from 50 nm to 100 nm.

The IPN preferably has a glass transition temperature (Tg) ranging from about −50° C. to +130° C. and preferably from −45° C. to +130° C.

The Tg is especially measured by differential scanning colorimetry (or DSC) using the DSC 7 machine from the company Perkin-Elmer, the polymer sample being preconditioned in a climatic chamber for 48 hours at 25° C. and 50% relative humidity, in an aluminum crucible.

The measurement is taken under a nitrogen flush, with a first heating ranging from −45° C. to +140° C. at a rate of 10° C./minute and a second heating ranging from −45° C. to +230° C.

IPNs are described in the publication Solvent-free urethane-acrylic hybrid polymers for coating; E. Galgoci et al., JCT Coatings Tech, 2(13), 28-36 (February 2005), and also in patents U.S. Pat. No. 4,644,030 and U.S. Pat. No. 5,173,526.

Preferably, the polymers are polymers of interpenetrating polymer network type comprising a polyurethane polymer and an acrylic polymer. Even more preferentially, the polymers are interpenetrating polymer networks of polyurethane and of acrylic polymer type in the form of an aqueous dispersion of particles.

Advantageously, the polyurethane/acrylic interpenetrating polymer network may be prepared according to the process described in patent U.S. Pat. No. 5,173,526. This process comprises the following steps: (a) forming a water-dispersible isocyanate-terminated polyurethane prepolymer comprising carboxylic groups; (b) adding to the prepolymer a mixture of vinyl monomer containing an ethylenically unsaturated monomer; (c) adding a tertiary amine to the prepolymer/vinyl monomer mixture; (d) dispersing the prepolymer/vinyl monomer mixture in water; (e) adding a radical initiator (soluble in oil) and a chain extender to the aqueous dispersion; and (f) polymerizing the vinyl monomers and completing the chain extension of the prepolymer by heating the aqueous dispersion.

The isocyanate-terminated polyurethane prepolymer may be obtained by reaction of organic monomer containing at least two active hydrogen atoms per molecule, especially a diol and preferably a polyester polyol, with an excess of diisocyanate monomer.

Preferably, the polyurethane prepolymer comprises unreacted carboxylic acid groups that are neutralized in tertiary amine salt form after the formation of the prepolymer and addition of the vinyl monomers, but before the formation of the aqueous dispersion.

The polyisocyanates used in the manufacture of the prepolymer may be aliphatic, cycloaliphatic or aromatic. Examples of polyisocyanates that may be mentioned include ethylene diisocyanate, hexamethylene 1,6-diisocyanate, isophorone diisocyanate, cyclohexane 1,4-diisocyanate, dicyclohexylmethane 4,4′-diisocyanate, phenylene 1,4-diisocyanate, toluene 2,4-diisocyanate, toluene 2,6-diisocyanate, diphenylmethane 4,4′-diisocyanate, diphenylmethane 2,4′-diisocyanate and naphthylene 1,5-diisocyanate, and mixtures thereof.

The polymeric polyols with a molecular weight ranging from 500 to 6000 and preferably ranging from 700 to 3000, which may be used for the preparation of the prepolymer, may be chosen from diols and triols, or mixtures thereof. The polyols may be chosen especially from polyesters, polyester amides, polyethers, polythioethers, polycarbonates and polyacetals.

The polyester polyols may be chosen from the hydroxyl-terminated reaction products of polyhydric alcohols such as ethylene glycol, propylene glycol, diethylene glycol, neopentyl glycol, 1,4-butanediol, furandimethanol, cyclohexanedimethanol, glycerol, trimethylolpropane or pentaerythritol, or mixtures thereof, with polycarboxylic acids, in particular dicarboxylic acids or the ester form thereof, such as succinic acid, glutaric acid, adipic acid or the methyl ester thereof, phthalic anhydride or dimethyl terephthalate. Polyesters obtained by polymerization of lactones, for instance caprolactone, and of polyol may also be used.

The polyesteramides may be obtained by using amino alcohols such as ethanolamine in the polyesterification mixture.

The polyether polyols that may be used include the products obtained by polymerization of cyclic oxide, for example ethylene oxide, propylene oxide, tetrahydrofuran, or by addition of these cyclic oxides to polyfunctional initiators such as water, ethylene glycol, propylene glycol, diethylene glycol, cyclohexanedimethanol, glycerol, trimethylolpropane, pentaerythritol or bisphenol A. The polyethers may also be chosen from polyoxypropylene diols and triols, poly(oxyethylene-oxypropylene) diols and triols obtained by simultaneous or sequential addition of propylene oxide and of ethylene oxide with suitable initiators, and polytetramethylene glycol ethers obtained by polymerization of tetrahydrofuran.

The polythioether polyols may be chosen from the products obtained by condensation of thiodiglycol, either alone or with other glycols, dicarboxylic acids, formaldehyde, amino alcohols or carboxylic amino acids.

The polycarbonate polyols may be chosen from the reaction products of diols such as 1,3-propanediol, 1,4-butanediol, 1,6-hexanediol, diethylene glycol or tetraethylene glycol with diaryl carbonates, for instance diphenyl carbonate, or with phosgene.

The polyacetal polyols may be chosen from the reaction products of glycols such as diethylene glycol, triethylene glycol or hexanediol with formaldehyde. The compounds bearing a reactive isocyanate group containing acid groups that may be used in the preparation of the water-dispersible anionic prepolymers comprise diols and triols containing carboxylic acid groups, for example those of formula (I):

R—C(CH₂OH)₂—COOH (I)

in which R is a hydrogen or a C₁-C₁₀ alkyl group. The diol bearing a carboxylic group is preferably 2,2-dimethylolpropionic acid. The diol or triol bearing a carboxylic group may be incorporated into a polyester by reaction with a dicarboxylic acid before being introduced into the prepolymer. Compounds bearing an acid group are, for example, aminocarboxylic acids, for example lysine, cystine or 3,5-diaminobenzoic acid.

The water-dispersible anionic isocyanate-terminated polyurethane prepolymer may be prepared in a conventional manner by reacting a stoichiometric excess of an organic polyisocyanate with a polymeric polyol and any other necessary compound that reacts with an isocyanate under anhydrous conditions at a temperature of between 30° C. and 130° C. until the reaction between the isocyanate groups and the hydroxyl groups is complete.

The polyisocyanate and the compounds containing an active hydrogen are advantageously used such that the ratio of the number of isocyanate groups to the number of hydroxyl groups ranges from 1.1/1 to 6/1 and preferably from 1.1/1 to 3/1. It is possible to use a well-known tin catalyst to assist the formation of the prepolymer.

A mixture of water-dispersible polyurethane prepolymer containing carboxylic groups and the vinyl monomer is obtained by simple addition of the vinyl monomer composition to the prepolymer. The vinyl monomer composition must contain at least one ethylenically unsaturated monomer.

The vinyl monomers that may be added to the prepolymer may be ethylenically unsaturated hydrocarbon-based monomers, ethylenically unsaturated esters, ethylenically unsaturated ethers, in particular (meth)acrylic acid esters, vinyl alcohol esters, or styrene.

Mention may be made especially of butadiene, isoprene, styrene, alkyl (meth)acrylates containing a C₁-C₆ alkyl group, alkyl maleates containing a C₁-C₆ alkyl group, vinyl acetate, vinyl butyrate, acrylonitrile, methyl vinyl ether, propyl vinyl ether, butyl vinyl ether, vinyl chloride and vinylidene chloride. The polyethylenically unsaturated monomers may be chosen from butadiene, isoprene, allyl methacrylate, diesters of acrylic acid and of C₂-C₆ diols such as butylene diacrylate and hexylene diacrylate, divinylbenzene, divinyl ether, divinyl sulfide and trimethylolpropane triacrylate.

Advantageously, the vinyl monomer is methyl methacrylate.

Before dispersing the prepolymer/vinyl monomer mixture in water, a tertiary amine is added to the mixture in an amount sufficient to make the prepolymer water-dispersible, i.e. in an amount sufficient to neutralize the carboxylic groups. For example, the amine may be added in an amount ranging from 65% to 100% of amine equivalent per equivalent carboxylic function.

The tertiary amines that may be used are relatively volatile, such that they are evaporated from the film after the film formation. Examples that may be mentioned include amines of formula R—N(R1)(R2) in which R, R1 and R2 independently represent a C₁-C₄ alkyl or hydroxyalkyl group. Mention may, for example, be made of triethylamine, dimethylethanolamine, methyldiethanolamine and methyldiethylamine.

It is important for the tertiary amine to be added to the mixture of prepolymer/monomers before this mixture is dispersed in water, in order to ensure compatibility of the organic and aqueous phases in the dispersion obtained.

The prepolymer/vinyl monomer mixture may be dispersed in water using the known techniques. Preferably, the mixture is added to water with stirring, or water may be poured into the mixture.

The chain extender containing the active hydrogen that reacts with the prepolymer may be a polyol, an amino alcohol, aqueous ammonia, a primary or secondary amine and, more particularly, a diamine.

Examples that may be mentioned include ethylenediamine, diethylenetriamine, triethylenetetramine, propylenediamine, butylenediamine, hexamethylenediamine, cyclohexylenediamine, piperazine, 2-methylpiperazine, phenylenediamine, toluenediamine, tris(2-aminoethyl)amine, 4,4′-methylenebis(2-chloroaniline), 3,3′-dichloro-4,4′-diphenyldiamine, 2,6-diaminopyridine, 4,4′-diaminodiphenylmethane and isophoronediamine.

The free-radical initiator may be an initiator of azo type such as 2,2′-azobis(2,4-dimethylpentanenitrile) and 2,2′-azobis(2-methylpropanenitrile) (or AIBN).

The radical polymerization of the mixture of vinyl monomers and the prepolymer chain extender is advantageously performed at high temperature, for example between 50° C. and 90° C. and preferably between 60° C. and 80° C.

The amount of chain extender used is advantageously equivalent to the free isocyanate groups in the prepolymer, the ratio of the number of active hydrogens in the chain extender to the number of isocyanate groups in the prepolymer preferably ranging from 0.7 to 1.3.

The polymerization of the vinyl monomers may be performed according to two methods. According to a first method, the monomers are added and may swell the polyurethane prepolymer before the addition of the tertiary amine. The monomers are then polymerized using the free-radical initiator. The proportion of the vinyl monomers may range from 25% to 75% by weight and preferably from 40% to 60% by weight relative to the total weight of solids in the aqueous dispersion.

According to a second polymerization method, part of the vinyl monomers is added to the prepolymer, the mixture is then neutralized with the tertiary amine and the prepolymer/vinyl monomer mixture is dispersed in water, followed by polymerization, during which the rest of the monomers is added. Alternatively, the second portion of monomers may be added to the prepolymer/vinyl monomer dispersion after addition of the amine, and the mixture stirred before the start of the polymerization.

The polymer dispersion may contain from 20% to 60% by weight of solids.

According to one preferred embodiment of the invention, the polyurethane present in the IPN is a copolymer of polyester polyol/diol with a carboxylic acid/diisocyanate/diamine group, such as those previously described for example; the acrylic polymer present in the IPN is a polymethyl methacrylate. The polyurethane/acrylic polymer IPN sold by the company Air Products under the trade name Hybridur® 875 Polymer Dispersion (INCI name: Polyurethane-2 (and) Polymethyl Methacrylate), or alternatively under the trade names Hybridur®) 870 and Hybridur® 880, is preferably used.

Grafted Silicone Polymers

For the purpose of the present invention, the term “grafted silicone polymer” means a polymer comprising a main chain of silicone 10 or polysiloxane (polymer of Si—O—) onto which is grafted, within said chain and also, optionally, at at least one of its ends, one or more organic groups not comprising silicone.

Examples of polymers with a polysiloxane backbone grafted with non-silicone organic monomers that are suitable for implementation of the present invention, and also the particular method for preparing same, are especially described in patent applications EP-A-0582152, WO 93/23009 and WO 95/03776, the teachings of which are entirely included in the present description by way of nonlimiting references.

According to a particularly preferred embodiment of the present invention, the silicone polymer with a polysiloxane backbone grafted with non-silicone organic monomers that is used comprises the result of radical copolymerization between, on the one hand, one or more non-silicone anionic organic monomers containing an ethylenic unsaturation and/or one or more non-silicone hydrophobic organic monomers containing an ethylenic unsaturation and, on the other hand, a silicone having in its chain one or more functional groups capable of reacting with said ethylenic unsaturations of said non-silicone monomers by forming a covalent bond, in particular thio functional groups.

According to the present invention, said ethylenically unsaturated anionic monomers are preferably chosen, alone or as mixtures, from neutralized unsaturated linear or branched carboxylic acids, it being possible for this or these unsaturated carboxylic acids to be more particularly acrylic acid, methacrylic acid, maleic acid, maleic anhydride, itaconic acid, fumaric acid and crotonic acid. It will be noted that, likewise, in the final grafted silicone polymer, the organic group of anionic nature which comprises the result of the radical (homo)polymerization of one or more anionic monomers of unsaturated carboxylic acid type.

For the purpose of the present invention, the term “hydrophobic monomer” means a monomer which has a solubility in water of less than 10 g per 100 mL of water at a temperature of 20° C.

According to the present invention, the ethylenically unsaturated hydrophobic monomers are preferably chosen, alone or as mixtures, from esters of acrylic acid of alkanols and/or esters of methacrylic acid of alkanols. The alkanols are preferably C₁-C₁₈ and more particularly C₁-C₁₂. The preferential monomers are chosen from the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, stearyl (meth)acrylate, or mixtures thereof.

A family of silicone polymers bearing a polysiloxane backbone grafted with non-silicone organic monomers that is particularly suitable for implementing the present invention consists of the silicone polymers comprising in their structure the unit of formula (II) below:

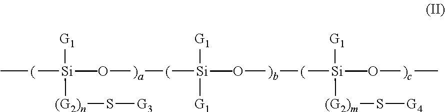

in which the radicals G, which may be identical or different, represent hydrogen or a C₁-C₁₀ alkyl radical or else a phenyl radical; the radicals G₂, which may be identical or different, represent a C₁-C₁₀ alkylene group; G₃ represents a polymeric residue resulting from the (homo)polymerization of at least one ethylenically unsaturated anionic monomer; G₄ represents a polymeric residue resulting from the (homo)polymerization of at least one ethylenically unsaturated hydrophobic monomer, m and n are, independently of each other, equal to 0 or 1; a is an integer ranging from 0 to 50; b is an integer that may be between 10 and 350, c is an integer ranging from 0 to 50; with the proviso that one of the parameters a and c is other than 0.

Preferably, the unit of formula (II) above features at least one, and even more preferentially all, of the following characteristics: the radicals G₁ denote a C₁-C₁₀ alkyl radical, n is non-zero and the radicals G₂ represent a divalent C₁-C₃ radical; G₃ represents a polymeric radical resulting from the (homo)polymerization of at least one monomer of the ethylenically unsaturated carboxylic acid type, preferably acrylic acid and/or methacrylic acid; G₄ represents a polymeric radical resulting from the (homo)polymerization of one or more monomers of the C₁-C₁₀ alkyl (meth)acrylate type.

Examples of grafted silicone polymers corresponding to formula (II) are thus in particular polydimethylsiloxanes (PDMS) onto which are grafted, by means of a thiopropylene-type chain unit, mixed polymer units of the poly((meth)acrylic acid) type and/or the poly(alkyl, in particular C₁-C₃, or even C₁, alkyl, (meth)acrylate) type. These polymers are referenced under the INCI name Polysilicone-8.

It may thus be a propylthio(poly(methyl acrylate/methyl methacrylate/methacrylic acid))-grafted polydimethylsiloxane or a propylthio(poly(methyl acrylate))-, propylthio(poly(methyl methacrylate))- and propylthio(poly(methacrylic acid))-grafted polydimethylsiloxane 10. As a variant, it may be a propylthio(poly(isobutyl methacrylate))- and propylthio(poly(methacrylic acid))-grafted polydimethylsiloxane.

Such grafted silicone polymers are sold especially by the company 3M under the trade names VS 80 and VS 70.

A propylthio(poly(methyl acrylate/methyl methacrylate/methacrylic acid))-grafted polydimethylsiloxane sold under the name VS 80 by the company 3M is preferably used.

Among the hydrophobic film-forming polymers in accordance with the invention, use will more preferentially be made of the polymers which are interpenetrating polymer networks (IPNs) of polyurethane and of acrylic polymer type in the form of an aqueous dispersion of particles, in particular the polyurethane/acrylic polymer IPN sold by the company Air Products under the trade name Hybridur® 875 Polymer Dispersion (INCI name: Polyurethane-2 (and) Polymethyl Methacrylate), or alternatively under the trade names Hybridur® 870 and Hybridur® 880.

Acrylic acid/N-tert-butylacrylamide Copolymers

Among the acrylic acid/N-tert-butylacrylamide copolymers, use will preferably be made of the non-neutralized acrylic acid/ethyl acrylate/N-tert-butylacrylamide copolymers (in which the acrylic acid is in free form) such as the products Ultrahold Strong and Ultrahold 8 (INCI name: Acrylates/t-Butylacrylanmide Copolymer) in non-neutralized form from the company BASF.

The term “non-neutralized (meth)acrylic acid/N-tert-butylacrylamide copolymer” means any (meth)acrylic acid/N-tert-butylacrylamide copolymer of which the (meth)acrylic acid function is free and is not neutralized with an organic or mineral base.

Vinyl Acetate/Crotonic Acid Copolymers

The term “non-neutralized crotonic acid/vinyl acetate copolymer means any crotonic acid/vinyl acetate copolymer of which the crotonic acid function is not neutralized with an organic or mineral base.

Among the non-neutralized vinyl acetate/crotonic acid copolymers, use will preferably be made of those described in patent FR 2 439 798, and in particular the vinyl acetate/crotonic acid/vinyl tert-butyl-4-benzoate copolymer (65/10/25) (INCI name: Vinyl acetate/vinyl butyl benzoate/crotonates copolymer) in non-neutralized form, such as the commercial product Mexomer PW manufactured by the company Chimex.

(Meth)acrylic acid Tetrapolymer

For the purpose of the present invention, the term “tetrapolymer” means a polymer derived from the copolymerization of four comonomers.

Among the tetrapolymers of (meth)acrylic acid, of (meth)acrylates and of C₈-C₂₄ alkyl (meth)acrylate, mention may be made of those described in patent application US2003021847, such as the copolymer sold under the name Soltex OPT-PG by the company Röhm & Haas, having the INCI name: Acrylates/C₁₂-C₂₂ Alkyl Methacrylate Copolymer.

Film-Forming Pseudoblocks

Among the film-forming pseudoblocks that are suitable for use in the invention, mention may be made of an acrylic acid/isobutyl acrylate/isobornyl acrylate copolymer sold under the name Mexomer PAS by the company Chimex, described in patent application FR 2 995 785.

Hydrocarbon-Based Block Copolymers

A hydrocarbon-based block copolymer that is suitable for use in the invention may especially be a diblock, triblock, multiblock, radial or star copolymer, or mixtures thereof.

Such hydrocarbon-based block copolymers are described in patent application US-A-2002/005562 and in patent U.S. Pat. No. 5,221,534.

The copolymer may contain at least one block whose glass transition temperature is preferably less than 20° C., preferably less than or equal to 0° C., preferably less than or equal to −20° C. and more preferably less than or equal to −40° C. The glass transition temperature of said block may be between −150° C. and 20° C. and especially between −100° C. and 0° C.

The hydrocarbon-based block copolymer present in the composition according to the invention is an amorphous copolymer formed by polymerization of an olefin. The olefin may especially be an elastomeric ethylenically unsaturated monomer.

Examples of olefins that may be mentioned include ethylenic carbide monomers, especially containing one or two ethylenic unsaturations and containing from 2 to 5 carbon atoms, such as ethylene, propylene, butadiene, isoprene or pentadiene.

Advantageously, the hydrocarbon-based block copolymer is an amorphous block copolymer of styrene and olefin.

Block copolymers comprising at least one styrene block and at least one block comprising units chosen from butadiene, ethylene, propylene, butylene and isoprene or a mixture thereof are especially preferred.

According to one preferred embodiment, the hydrocarbon-based block copolymer is hydrogenated to reduce the residual ethylenic unsaturations after the polymerization of the monomers.

In particular, the hydrocarbon-based block copolymer is an optionally hydrogenated copolymer, containing styrene blocks and ethylene/C₃-C₄ alkylene blocks.

According to one preferred embodiment, the composition according to the invention comprises at least one diblock copolymer, which is preferably hydrogenated, preferably chosen from styrene-ethylene/propylene copolymers, styrene-ethylene/butadiene copolymers and styrene-ethylene/butylene copolymers. Diblock polymers are especially sold under the name Kraton® G1701E by the company Kraton Polymers.

According to another preferred embodiment, the composition according to the invention comprises at least one triblock copolymer, which is preferably hydrogenated, preferably chosen from styrene-ethylene/propylene-styrene copolymers, styrene-ethylene/butadiene-styrene copolymers, styrene-isoprene-styrene copolymers and styrene-butadiene-styrene copolymers. Triblock polymers are especially sold under the names Kraton® G1650, Kraton® D1101, Kraton® D1102 and Kraton® D1160 by the company Kraton Polymers.

According to one embodiment of the present invention, the hydrocarbon-based block copolymer is a styrene-ethylene/butylene-styrene triblock copolymer.

According to one preferred embodiment of the invention, it is especially possible to use a mixture of a styrene-butylene/ethylene-styrene triblock copolymer and of a styrene-ethylene/butylene diblock copolymer, especially the products sold under the name Kraton® G1657M by the company Kraton Polymers.

According to another preferred embodiment, the composition according to the invention comprises a mixture of styrene-butylene/ethylene-styrene hydrogenated triblock copolymer and of ethylene-propylene-styrene hydrogenated star polymer, such a mixture possibly being especially in isododecane or in another oil. Such mixtures are sold, for example, by the company Penreco under the trade names Versagel® M5960 and Versagel® M5670.

According to a preferred embodiment, a hydrophobic film-forming polymer in accordance with the invention is chosen from:
a diblock copolymer, which is preferably hydrogenated, preferably chosen from styrene-ethylene/propylene copolymers, styrene-ethylene/butadiene copolymers and styrene-ethylene/butylene copolymers,
a film-forming pseudoblock, preferably an acrylic acid/isobutyl acrylate/isobornyl acrylate copolymer, and
mixtures thereof.

The hydrophobic film-forming polymers that are suitable for use in the invention may be soluble or dispersed in the fatty phase when it is present, in an amount of between 1% and 10% by weight of active material, preferably between 2% and 8% by weight and even more preferentially between 3% and 6% by weight of active material relative to the total weight of the composition.

Hydrophilic Film-Forming Polymers

The term “hydrophilic film-forming polymer” or “aqueous film-forming polymer” means any polymer:
(1) which is capable of forming, by itself or in the presence of an auxiliary film-forming agent, a continuous or discontinuous film that adheres to a support, especially to the skin; and
(2) for which the film formed is capable of absorbing an amount of water greater than 35%, preferably greater than or equal to 40% and more particularly greater than or equal to 60% by weight relative to the weight of the dry polymer film (before immersion in water) when it is dipped into liquid water.

The amount of water absorbed by the hydrophilic polymers that are suitable for use in the present invention can be measured under the same conditions as those described for the hydrophobic polymers.

The hydrophilic polymer(s) used according to the invention are film-forming polymers that are capable of forming, by themselves or in the presence of an auxiliary film-forming agent, a continuous film capable of adhering to a support, especially to the skin.

Needless to say, a person skilled in the art will choose the polymers as a function of their compatibility with the compound chosen from an aluminum and/or zirconium salt, an aluminum and/or zirconium complex, and mixtures thereof used in a composition according to the invention.

Preferably, the hydrophilic film-forming polymer(s) are chosen from polyurethanes, vinyl polymers, natural polymers, latices and pseudolatices, and mixtures thereof.

Hydrophilic Film-Forming Polyurethanes

The polyurethanes may be aliphatic, cycloaliphatic or aromatic polyurethane, polyurea-urethane or polyurea copolymers, comprising, alone or as a mixture:
at least one block of aliphatic and/or cycloaliphatic and/or aromatic polyester origin, and/or
at least one branched or unbranched silicone block, for example polydimethylsiloxane or polymethylphenylsiloxane, and/or
at least one block comprising fluoro groups.

The film-forming polyurethanes that can be used in the invention may also be obtained from branched or non-branched polyesters or from alkyls comprising labile hydrogens, which are modified by reaction with a diisocyanate and a difunctional organic compound (for example dihydroxy, diamino or hydroxyamino), also comprising either a carboxylic acid or carboxylate group, or a sulfonic acid or sulfonate group, or alternatively a neutralizable tertiary amine group or a quaternary ammonium group.

With a view to forming the polyurethane, monomers bearing an anionic group that can be used during the polycondensation that maybe mentioned include dimethylolpropionic acid, trimellitic acid or a derivative such as trimellitic anhydride, the sodium salt of 3-sulfopentanediol acid, and the sodium salt of 5-sulfo-1,3-benzenedicarboxylic acid.

Preferably, the monomer bearing an anionic group is dimethylolpropionic acid.

As film-forming polyurethane that may be used according to the invention, mention may thus be made of the aqueous polyurethane dispersions sold under the names Avalure UR-405®, Avalure UR-410®, Avalure UR-425® and Avalure UR-450® by the company Goodrich.

The hydrophilic film-forming polyurethanes may also be chosen from film-forming elastomeric polyurethanes capable of producing, by drying of said polyurethane(s), at room temperature and at a relative humidity of 55%, a material having a mechanical profile defined by at least:

a) a degree of elongation at break (ε) of greater than or equal to 150%,

b) an instantaneous recovery (Rᵢ) of greater than or equal to 75% after an elongation of 150%,

c) a recovery at 300 seconds (R₃₀₀ₛ) of greater than 80%, after an elongation of 150%.

The material obtained by drying said film-forming polyurethane(s) is therefore sufficiently extensible so as not to break following the deformations caused by the movements of the skin and to regain a shape substantially identical to its initial shape.

For the purpose of the present invention, the instantaneous recovery (Rᵢ) of a material defines the capacity of said material to regain its initial shape or a shape substantially identical to its initial shape after having been deformed following an elongation during a tensile stress. The recovery of the material is also measured as a percentage.

For the purposes of the present invention, the degree of elongation at break and the recovery are evaluated by means of the tensile tests described below. To perform the tensile tests, a film intended for producing specimens is made by placing in a Teflon mold a sufficient amount of mixture comprising the film-forming elastomeric polymer(s) to obtain a film 500 μm±50 μm thick. Drying is continued until the weight of the film no longer changes, which may typically take 12 days.

In particular, for the purpose of the present invention, the expression “film intended for preparing or producing test specimens” means a film obtained by drying said film-forming elastomeric polyurethane(s), at room temperature (22° C.±2° C.) and at a relative humidity of 55%±5%, from a mixture containing at least 3% of active materials, i.e. 3% by weight of polyurethanes relative to the total weight of the mixture.

When the mixture used to produce the film for the manufacture of specimens contains less than 3% by weight of active materials, a preliminary concentration operation is performed, for example by evaporating off some of the solvent so that the mixture contains at least 3% of elastomeric polymers. This operation makes it possible to avoid excessively long drying. The film obtained is then chopped into rectangular specimens 80 mm long and 15 mm wide.

The tests are performed on a machine sold under the name Lloyd or sold under the name Zwick, under the same temperature and humidity conditions as for the drying, i.e. room temperature (22° C.±2° C.) and at a relative humidity of 55%±5%. The specimens are drawn at a rate of 20 mm/minute and the distance between the jaws is 50±1 mm.

To determine the instantaneous recovery (Rᵢ), the following process is performed:
the specimen is drawn by 150% (εₘₐₓ), i.e. 1.5 times its initial length (Iₒ),
the stress is removed by imposing a return speed equal to the tensile speed, i.e. 20 mm/minute, and the elongation of the specimen is measured as a percentage, after returning to zero load (εᵢ).

The % instantaneous recovery (Rᵢ) is given by the following formula:

Rᵢ=((εₘₐₓ−εᵢ)/εₘₐₓ)×100

To determine the recovery at 300 seconds, the specimen is maintained at zero stress for a further 300 seconds, after having undergone the preceding operations, and its degree of elongation is measured as a percentage (C₃₀₀ₛ). In other words, the recovery at 300 seconds corresponds to the residual degree of elongation of the specimen 300 seconds after returning to zero load (εᵢ).

Thus, the recovery at 300 seconds (R₃₀₀ₛ) of a material defines the capacity of said material to regain its shape or a shape substantially identical to its initial shape after a further 300 seconds after the return to zero load (εᵢ) and after having been deformed following an elongation during a tensile stress.

The percentage recovery at 300 seconds (R₃₀₀ₛ) is therefore given by the formula below:

R₃₀₀ₛ=((εₘₐₓ−εᵢ)/ε₃₀₀ₛ)×100

Advantageously, the film-forming elastomeric polyurethane(s) that are suitable for use in the invention are such that they form, under the conditions of the tests described above, a material having a degree of elongation at break (ε) greater than 150%, preferably at least greater than 250%, and even more preferentially ranging from 250% to 1000%, an instantaneous recovery (Rᵢ) ranging from 75% to 100% and a recovery at 300 seconds (R₃₀₀ₛ) ranging from 80% to 100%, preferably from 90% to 100%.

Preferably, the film-forming elastomeric polyurethanes are chosen from copolymers obtained by copolymerization of hexanediol, neopentyl glycol, adipic acid, hexamethylene diisocyanate, N-(2-aminoethyl)-3-aminoethanesulfonic acid and ethylenediamine.

Preferably, the polyurethanes may also be chosen from copolymers obtained by copolymerization of adipic acid, dicyclohexylmethane diisocyanate, ethylenediamine, hexanediol, neopentyl glycol and sodium N-(2-aminoethyl)-3-aminoethanesulfonate.

In particular, the polyurethanes are chosen from those sold under the name Baycusan C1001 or C1004, and more particularly the product sold under the name Baycusan C1001.

Hydrophilic Film-Forming Vinyl Polymers

Preferably, the vinyl polymer(s) are chosen from polyvinyl alcohols, copolymers derived from C₄-C₈ monounsaturated carboxylic acids or anhydrides, and methyl vinyl ether/butyl monomaleate copolymers. For the purpose of the present invention, the term “polyvinyl alcohol” means a polymer comprising —CH₂CH(OH)— units.

The polyvinyl alcohols are generally produced by hydrolysis of polyvinyl acetate. Usually, the reaction takes place in the presence of methanol (alcoholysis). The reaction is normally catalyzed by acidic or basic catalysis. The degree of hydrolysis of the commercial products is variable, often around 87%, but products with a 100 degree of hydrolysis also exist. Copolymers with monomers other than vinyl acetate also exist, such as ethylene/vinyl alcohol copolymers.

The polyvinyl alcohol polymers are preferably chosen from homopolymers or copolymers with vinyl acetate, the latter corresponding in particular to a partial hydrolysis of polyvinyl acetate.

Use may, for example, be made of the products of the Celvol range provided by the company Celanese under the names Celvol 540, Celvol 350, Celvol 325, Celvol 165, Celvol 125, Celvol 540 S, Celvol 840 and Celvol 443.

Preferably, the polyvinyl alcohols are chosen from the products sold under the name Celvol 540 by the company Celanese.

The copolymer(s) derived from C₄-C₈ monounsaturated carboxylic acids or anhydrides may be chosen from copolymers comprising (i) one or more maleic, fumaric or itaconic acids or anhydrides and (ii) one or more monomers chosen from vinyl esters, vinyl ethers, vinyl halides, phenylvinyl derivatives, and acrylic acid and its esters, the anhydride functions of these copolymers being optionally monoesterified or monoamidated.

Such polymers are described in particular in patents U.S. Pat. No. 2,047,398, U.S. Pat. No. 2,723,248 and U.S. Pat. No. 2,102,113 and patent GB 839 805, and in particular those sold under the names Gantrez AN or ES and Avantage CP by the company ISP, having the INCI name: Butyl Ester of PVM/MA Copolymer.

Preferably, the copolymer(s) derived from C₄-C₈ monounsaturated carboxylic acids or anhydrides are chosen from the monoesterified methyl vinyl ether/maleic anhydride copolymers sold, for example, under the name Gantrez ES 225 by the company ISP.

Natural Hydrophilic Film-Forming Polymers

Needless to say, a person skilled in the art will choose the polymers as a function of their compatibility with the compound chosen from an aluminum and/or zirconium salt, an aluminum and/or zirconium complex, and mixtures thereof used in a composition according to the invention.

The hydrophilic polymer(s) may also be chosen from natural polymers, in particular polysaccharides which have monosaccharides or disaccharides as base units.

The natural polymers are preferably chosen from guar gums and modified guar gums, celluloses, and gellan gum and derivatives thereof.

Guar gums are galactomannans consisting of mannose and galactose.

For the purpose of the present invention, the term “modified guar gum” means guar gums alkylated with at least one C₁-C₈ alkyl group, guar gums hydroxyalkylated with at least one C₁₋₈ hydroxyalkyl group and guar gums acylated with at least one C₁₋₈ acyl group.

Preferably, they are hydroxypropylated guar gums such as the product sold under the name Jaguar HP 105 by the company Rhodia.

The cellulose is a β1-4-polyacetal of cellobiose, cellobiose being a disaccharide consisting of two glucose molecules.

The cellulose derivatives may be cationic, amphoteric or nonionic. Among these derivatives, cellulose ethers, cellulose esters and cellulose ester ethers are distinguished. Among the nonionic cellulose ethers, mention may be made of alkylcelluloses such as methylcelluloses and ethylcelluloses; hydroxyalkylcelluloses such as hydroxymethylcelluloses, hydroxyethylcelluloses and hydroxypropylcelluloses; and mixed hydroxyalkyl-alkylcelluloses such as hydroxypropylmethylcelluloses, hydroxyethylmethylcelluloses, hydroxyethylethylcelluloses and hydroxybutylmethylcelluloses.

Among the cationic cellulose ethers, mention may be made of crosslinked or non-crosslinked quaternized hydroxyethylcelluloses. The quaternizing agent may especially be glycidyltrimethylammonium chloride or a fatty amine such as laurylamine or stearylamine. Another cationic cellulose ether that may be mentioned is hydroxyethylcellulosehydroxypropyltrimethylammonium. Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates.

Among the cellulose ester ethers, mention may be made of hydroxypropylmethylcellulose phthalates and ethylcellulose sulfates. The cellulose-based compounds of the invention may be chosen from unsubstituted celluloses and substituted celluloses.

The celluloses and derivatives are represented, for example, by the products sold under the names Avicel® (microcrystalline cellulose, MCC) by the company FMC Biopolymers, under the name Methocel™ (cellulose ethers) and Ethocel™ (ethylcellulose) by the company Dow, Benecel® (methylcellulose), Blanose™ (carboxymethylcellulose), Culminai® (methylcellulose, hydroxypropylmethylcellulose), Klucel® (hydroxypropylcellulose), Polysurf® (cetylhydroxyethylcellulose) and Natrosol® CS (hydroxyethylcellulose) by the company Hercules Aqualon.

Gellan gum is a polysaccharide produced by aerobic fermentation of Sphingomonas elodea, more commonly known as Pseudomonas elodea. This linear polysaccharide is formed from the sequence of the following monosaccharides: D-glucose, D-glucuronic acid and L-rhamnose.

In native form, gellan gum is highly acylated.

The gellan gum preferably used in the film according to the present invention is a gellan gum that is at least partially deacylated. This at least partially deacylated gellan gum is obtained by high-temperature alkaline treatment. A solution of KOH or of NaOH will, for example, be used. The purified gellan gum sold under the trade name Kelcogel® by the company Kelco is suitable for preparing the compositions according to the invention.

Gellan gum derivatives are all the products obtained by performing standard chemical reactions, especially such as esterifications, addition of a salt of an organic or mineral acid. Welan gum is used, for example, as a gellan gum derivative. Welan gum is a gellan gum modified by fermentation by means of Alcaligenes strain ATCC 31 555. Welan gum has a recurring pentasaccharide structure formed from a main chain consisting of D-glucose, D-glucuronic acid and L-rhamnose units, onto which a pendent L-rhamnose or L-mannose unit is grafted.

The welan gum sold under the trade name Kelco Crete® by the company Kelco is suitable for preparing the compositions according to the invention.

As other saccharide polymers that can be used according to the invention, mention may be made of starches and derivatives thereof.

Preferably, the natural polymer(s) are chosen from celluloses and derivatives thereof, in particular those sold under the name Avicel® (microcrystalline cellulose, MCC) by the company FMC Biopolymers.

The hydrophilic polymers may also be chosen from acrylate and methacrylate copolymers.

Preferably, the hydrophilic polymer(s) are chosen from the polyurethanes sold under the name Baycusan C1004 and Baycusan C1001 by the company Bayer Material Science.

Latex or Pseudolatex

As presented above for the hydrophilic film-forming polyurethanes, the hydrophilic film-forming polymer may thus also be present in a composition of the invention in the form of particles dispersed in an aqueous phase, which is generally known as a latex or pseudolatex. Techniques for preparing these dispersions are well known to those skilled in the art.

Aqueous dispersions of film-forming polymers that may be used include the acrylic dispersions sold under the names Neocryl XK-90, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® or Syntran PC 5100® by the company Interpolymer, Allianz OPT by the company Röhm & Haas, aqueous dispersions of acrylic or styrene/acrylic polymers sold under the brand name Joncryl® by the company Johnson Polymer, or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer; the sulfopolyesters sold under the brand name Eastman AQ® by the company Eastman Chemical Products, and vinyl dispersions, for instance Mexomer PAM® from the company Chimex, and mixtures thereof.

Preferably, the hydrophilic film-forming polymer that is suitable for use in the invention is chosen from a latex, a pseudolatex, a polyurethane, and mixtures thereof, and preferably from a latex, a pseudolatex, and mixtures thereof, and even more preferentially a hydrophilic film-forming polymer that is suitable for use in the invention is a latex such as an acrylate copolymer.

The hydrophilic film-forming polymers that are suitable for use in the invention are present in an amount of between 0.1% and 20% by weight of active material, preferably between 3% and 15% by weight and even more preferentially between 4% and 11% by weight of active material relative to the total weight of the composition.

Thus, the film-forming polymer(s) that are suitable for use in the invention are present in a content of between 0.1% and 40% by weight of active material, preferably between 1% and 30% by weight of active material, even more preferentially between 2% and 20% by weight and better still between 4% and 15% by weight of active material relative to the total weight of the composition.

Physiologically Acceptable Medium

As presented above, a composition according to the invention may advantageously be a cosmetic or dermatological composition.

In this particular embodiment, since a composition according to the invention is intended for topical application to the skin and/or the nails, it contains a physiologically acceptable medium.

For the purposes of the present invention, the term “physiologically acceptable medium” means a medium that is compatible with the skin and/or the nails.

Thus, the physiologically acceptable medium is in particular a cosmetically or dermatologically acceptable medium, i.e. a medium that has no unpleasant odor, color or appearance, and that does not cause the user any unacceptable stinging, tautness or redness.

Aqueous Phase

The aqueous phase of a composition according to the invention comprises water and optionally a water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

Among the water-soluble solvents that may be used in the composition in accordance with the invention, mention may be made especially of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

The aqueous phase (water and optionally the water-miscible solvent) may be present in the composition in a content ranging from 5% to 95%, better still from 30% to 80% by weight and preferably from 40% to 75% by weight relative to the total weight of said composition.

According to another embodiment variant, the aqueous phase of a composition according to the invention may comprise at least one C₂-C₃₂ polyol.

For the purposes of the present invention, the term “polyol” should be understood as meaning any organic molecule comprising at least two free hydroxyl groups.

Preferably, a polyol in accordance with the present invention is present in liquid form at room temperature.

A polyol that is suitable for use in the invention may be a compound of linear, branched or cyclic saturated or unsaturated alkyl type, bearing on the alkyl chain at least two —OH functions, in particular at least three —OH functions and more particularly at least four —OH functions.

The polyols that are advantageously suitable for formulating a composition according to the present invention are those especially containing from 2 to 32 carbon atoms and preferably 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols such as glycerol oligomers, for instance diglycerol, and polyethylene glycols, and mixtures thereof.

According to a preferred embodiment of the invention, said polyol is chosen from ethylene glycol, pentaerythritol, trimethylolpropane, propylene glycol, glycerol, polyglycerols, polyethylene glycols and mixtures thereof.

According to a particular mode, the composition of the invention may comprise at least propylene glycol.

According to another particular mode, the composition of the invention may comprise at least glycerol.

Depending on the presentation form or, when the composition is in the form of an emulsion, depending on the sense of the emulsion, the aqueous phase may be composed of a synthetic phyllosilicate that is suitable for use in the invention in gel form, alone or in combination with other gelling agents.

As presented above, according to a particular embodiment, a synthetic phyllosilicate that is suitable for use in the invention may be used in the form of an aqueous or aqueous-alcoholic gel. When the gel is aqueous, it may then constitute all or part of the aqueous phase.

Thus, according to a particular embodiment, a synthetic phyllosilicate that is suitable for use in the invention in aqueous gel form constitutes the aqueous phase of a composition according to the invention, i.e. the aqueous phase of the emulsion is exclusively constituted of this aqueous gel.

Fatty Phase

For the purposes of the invention, a fatty phase includes any liquid fatty substance, generally oils (also known as oily or liquid fatty phase), or solid fatty substances like waxes or pasty compounds (also known as solid fatty phase).

For the purposes of the invention, a liquid fatty phase comprises at least one oil.

The term “oil” means any fatty substance that is in liquid form at room temperature and atmospheric pressure.

Needless to say, when the composition according to the invention comprises a film-forming agent chosen from a hydrophilic film-forming polymer, a hydrophobic film-forming polymer and mixtures thereof, the oils of the composition of the invention are chosen from oils that are compatible with these film-forming agents.

An oily phase that is suitable for preparing the cosmetic compositions according to the invention may comprise hydrocarbon-based oils, silicone oils, fluoro oils or non-fluoro oils, or mixtures thereof.

The oils may be volatile or non-volatile.

They may be of animal, plant, mineral or synthetic origin. According to one embodiment, oils of plant origin are preferred.

For the purposes of the present invention, the term “nonvolatile oil” means an oil with a vapor pressure of less than 0.13 Pa.

For the purposes of the present invention, the term “silicone oil” means an oil comprising at least one silicon atom, and in particular at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, especially having a nonzero vapor pressure, at room temperature and atmospheric pressure, especially having a vapor pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Volatile Oils

The volatile oils may be hydrocarbon-based oils or silicone oils.

Among the volatile hydrocarbon-based oils containing from 8 to 16 carbon atoms, mention may be made especially of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof. Preferably, the volatile hydrocarbon-based oil is selected from volatile hydrocarbon-based oils containing from 8 to 16 carbon atoms, and mixtures thereof, in particular from isododecane, isodecane and isohexadecane, and is especially isohexadecane.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in examples 1 and 2 of patent application WO 2008/155 059 from the company Cognis, and mixtures thereof.

Volatile silicone oils that may be mentioned include linear volatile silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane and dodecamethylcyclohexasiloxane.

Nonvolatile Oils

The nonvolatile oils may, in particular, be chosen from non-volatile hydrocarbon-based, fluoro and/or silicone oils.

Nonvolatile hydrocarbon-based oils that may in particular be mentioned include:
hydrocarbon-based oils of animal origin,
hydrocarbon-based oils of plant origin, synthetic ethers containing from 10 to 40 carbon atoms, such as dicaprylyl ether,
synthetic esters, such as the oils having formula R₁COOR₂, in which R₁ represents a linear or branched fatty acid residue comprising from 1 to 40 carbon atoms and R₂ represents a hydrocarbon-based chain, which is in particular branched, containing from 1 to 40 carbon atoms, on condition that R₁+R₂ is ≧10. The esters may be chosen especially from fatty acid alcohol esters, for instance cetostearyl octanoate, isopropyl alcohol esters such as isopropyl myristate or isopropyl palmitate, ethyl palmitate, 2-ethylhexyl palmitate, isopropyl stearate, octyl stearate, hydroxylated esters, such as isostearyl lactate or octyl hydroxystearate, alcohol or polyalcohol ricinoleates, hexyl laurate, neopentanoic acid esters, such as isodecyl neopentanoate or isotridecyl neopentanoate, and isononanoic acid esters, such as isononyl isononanoate or isotridecyl isononanoate,
polyol esters and pentaerythritol esters, such as dipentaerythrityl tetrahydroxystearate/tetraisostearate,
fatty alcohols that are liquid at room temperature, with a branched and/or unsaturated carbon-based chain containing from 12 to 26 carbon atoms, for instance 2-octyldodecanol, isostearyl alcohol and oleyl alcohol,
C₁₂-C₂₂ higher fatty acids, such as oleic acid, linoleic acid, linolenic acid, and mixtures thereof,
non-phenyl silicone oils, for instance caprylyl methicone, and
phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, and trimethyl-pentaphenyl-trisiloxane, and mixtures thereof; and also mixtures of these various oils.

Preferably, a composition according to the invention comprises volatile and/or non-volatile silicone oils.

A composition according to the invention may comprise from 5% to 95% by weight, better still from 5% to 40% by weight, and preferably from 7% to 35% by weight of oil(s) relative to the total weight of said composition.

Waxes

For the purposes of the present invention, the term “wax” means a lipophilic fatty compound that is solid at room temperature (25° C.), with a reversible solid/liquid change of state, having a melting point of greater than 30° C. which may be up to 200° C., a hardness of greater than 0.5 MPa, and having an anisotropic crystal organization in the solid state. By bringing the wax to its melting point, it is possible to make it miscible with oils and to form a microscopically homogeneous mixture, but on returning the temperature of the mixture to room temperature, recrystallization of the wax in the oils of the mixture is obtained.

The waxes that may be used in the invention are compounds that are solid at room temperature, which are intended to structure the composition in particular in stick form; they may be hydrocarbon-based, fluoro and/or silicone-based and may be of plant, mineral, animal and/or synthetic origin. In particular, they have a melting point of greater than 40° C. and better still greater than 45° C.

As waxes that may be used in the invention, mention may be made of those generally used in cosmetics: they are especially of natural origin, such as beeswax, carnauba wax, candelilla wax, ouricury wax, Japan wax, cork fiber wax or sugarcane wax, rice wax, montan wax, paraffin wax, lignite wax or microcrystalline wax, ceresin or ozokerite, hydrogenated oils such as jojoba oil; synthetic waxes such as polyethylene waxes derived from the polymerization or copolymerization of ethylene and Fischer-Tropsch waxes, or alternatively fatty acid esters such as octacosanyl stearate, glycerides that are concrete at 40° C. and better still at 45° C., silicone waxes such as alkyl or alkoxy dimethicones with an alkyl or alkoxy chain of 10 to 45 carbon atoms, poly(di)methylsiloxane esters that are solid at 40° C., the ester chain of which comprises at least 10 carbon atoms; and mixtures thereof.

As a guide, a composition according to the invention may comprise from 0.01% to 50% by weight, preferably from 2% to 40% by weight, and better still from 5% to 30% by weight of wax(es), relative to the total weight of the composition.

Pasty Compound

For the purposes of the present invention, the term “pasty” is intended to denote a lipophilic fatty compound with a reversible solid/liquid change of state, and comprising at a temperature of 23° C. a liquid fraction and a solid fraction.

The pasty compound is advantageously chosen from:
lanolin and derivatives thereof,
polymeric or non-polymeric fluoro compounds,
polymeric or non-polymeric silicone compounds,
vinyl polymers, especially:
olefin homopolymers,
olefin copolymers,
hydrogenated diene homopolymers and copolymers,
linear or branched homopolymer or copolymer oligomers of alkyl (meth)acrylates preferably bearing a C₈-C₃₀ alkyl group,
homopolymer and copolymer oligomers of vinyl esters bearing C₈-C₃₀ alkyl groups,
homopolymer and copolymer oligomers of vinyl ethers bearing C₈-C₃₀ alkyl groups,
liposoluble polyethers resulting from polyetherification between one or more C₂-C₁₀₀ and preferably C₂-C₅₀ diols,
esters,
polyvinyl laurate; and
mixtures thereof.

As a guide, a composition according to the invention may comprise from 1% to 99% by weight, preferably from 1% to 60% by weight, better still from 2% to 30% by weight and better still from 5% to 20% by weight of pasty compound(s), relative to the total weight of the composition.

In the case of emulsions, the proportion of fatty phase will be chosen according to the sense of the emulsion.

The fatty phase may thus be present in the composition in an amount ranging from 1% to 80%, better ranging still from 5% to 70% and even better still from 10% to 60% by weight relative to the total weight of the composition.

Preferably, the fatty phase comprises an oil chosen from alkanes, such as isohexadecane and isododecane, esters such as isopropyl palmitate, ethers such as dicaprylyl ether, triglycerides such as capric/caprylic acid triglyceride and silicones, specifically nonvolatile silicones such as polydimethylsiloxanes, such as PDMS 6 cSt.

As oil that may be used in the composition according to the invention, mention may also be made of the hydrogenated polyisobutene sold especially by the company Nippon Oil under the name Parleam®.

Additives

A composition according to the invention may also comprise one or more additional agents chosen from antioxidants, preserving agents, UV-screening agents, thickeners, fragrances, neutralizers, spreading agents, antifoams, dispersants, stabilizers, surfactants, gelling agents, sequestrants, softeners, wetting agents, opacifiers, emollients, silicones, fillers other than a synthetic phyllosilicate according to the invention, polymers, fragrances, propellants, acidifying or basifying agents or any other ingredient usually used in the cosmetic and/or dermatological field, and mixtures thereof.

Similarly, a composition according to the invention may also comprise at least one dyestuff chosen, for example, from pigments, nacres, dyes and materials with an effect, and mixtures thereof.

These dyestuffs may be present in a content ranging from 0.01% to 50% by weight and preferably from 0.01% to 30% by weight relative to the total weight of the composition.

In addition, a composition according to the invention may also comprise at least one active agent chosen from moisturizers, cicatrizing agents and/or antiaging agents for the skin.

When the electrolyte is chosen from an aluminum and/or zirconium salt, an aluminum and/or zirconium complex, and mixtures thereof, preferably chosen from aluminum halohydrates, aluminum zirconium halohydrates, complexes of zirconium hydroxychloride and of aluminum hydroxychloride, with or without an amino acid, and mixtures thereof, and also comprising (c) at least one film-forming polymer chosen from a hydrophilic film-forming polymer and a hydrophobic film-forming polymer, and mixtures thereof, a composition according to the invention may also comprise additional active agents such as additional deodorant active agents.

The term “deodorant active agent” refers to any substance that is capable of masking, absorbing, improving and/or reducing the unpleasant odor resulting from the decomposition of human sweat by bacteria.

The deodorant active agents may be bacteriostatic agents or bactericides that act on underarm odor microorganisms, such as 2,4,4′-trichloro-2′-hydroxydiphenyl ether (Triclosan), 2,4-dichloro-2′-hydroxydiphenyl ether, 3′,4′,5′-trichlorosalicylanilide, 1-(3′,4′-dichlorophenyl)-3-(4′-chlorophenyl)urea (Triclocarban) or 3,7,11-trimethyldodeca-2,5,10-trienol (Farnesol); quaternary ammonium salts such as cetyltrimethylammonium salts, cetylpyridinium salts, polyols such as those of glycerol type, 1,3-propanediol (especially Zemea Propanediol® sold by the company DuPont Tate & Lyle Bio Products), 1,2-decanediol (sold especially under the trade name Symclariol® by the company Symrise), glycerol derivatives, for instance caprylic/capric glycerides (sold especially under the trade name Capmul MCM® by the company Abitec), glyceryl caprylate or caprate (sold especially under the trade names Dermosoft GMCY® and Dermosoft GMC®, respectively by the company Straetmans), polyglyceryl-2 caprate (sold especially under the trade name Dermosoft DGMC® by the company Straetmans), biguanide derivatives, for instance polyhexamethylene biguanide salts; chlorhexidine and salts thereof; 4-phenyl-4,4-dimethyl-2-butanol (sold especially under the trade name Symdeo MPP® by the company Symrise); cyclodextrins; chelating agents such as those sold under the trade name Dissolvine GL-47-S® by the company AkzoNobel, EDTA and DPTA (1,3-diaminopropanetetraacetic acid).

Among the deodorant active agents in accordance with the invention, mention may also be made of: —zinc salts, for instance zinc salicylate, zinc gluconate, zinc pidolate; zinc sulfate, zinc chloride, zinc lactate, zinc phenolsulfonate; zinc ricinoleate;
sodium bicarbonate;
salicylic acid and derivatives thereof such as 5-n-octanoylsalicylic acid;
zeolites, especially silver-free metallic zeolites;
alum; and
triethyl citrate.

Thus, according to a particular embodiment, a composition according to the invention comprises at least one additional deodorant active agent and/or one additional antiperspirant active agent.

The deodorant active agents may preferably be present in the compositions according to the invention in weight proportions ranging from 0.01% to 10% by weight relative to the total weight of the composition.

Needless to say, all the abovementioned additional agents or compounds are different from the synthetic phyllosilicates and from the electrolytes and polyelectrolytes described previously.

Needless to say, a person skilled in the art will take care to select the optional additional ingredients or compounds and/or the amount thereof, especially among those mentioned above, for the purpose of the intended use, or alternatively such that the advantageous properties of the composition according to the invention are not, or are not substantially, adversely affected by the envisaged addition.

The additives are generally present in the composition according to the invention in an amount ranging from 0% to 20% by weight relative to the total weight of the composition.

Composition

The compositions according to the invention may be prepared according to the techniques that are well known to those skilled in the art.

The composition according to the invention may be in any presentation form conventionally used for the intended application.

For example, when the composition according to the invention is cosmetic or dermatological, it may be in any presentation form conventionally used for topical applications and especially in the form of a dispersion of aqueous gel or lotion type, of an emulsion of liquid to semisolid consistency, obtained by dispersing a fatty phase in an aqueous phase (O/W) or conversely (W/O), or of a liquid to semisolid suspension of emulsified gel or cream type.

Preferably, the composition is in the form of an oil-in-water (direct emulsion (O/W)) or water-in-oil (inverse emulsion (W/O)) emulsion, preferably an oil-in-water emulsion, a gel or an emulsified gel. The emulsions may contain stabilizers, for instance fillers other than a synthetic phyllosilicate that is suitable for use in the invention, gelling polymers or thickening polymers, as presented above.

As mentioned above, a synthetic phyllosilicate that is suitable for use in the invention is in the form of a gel and more particularly of an aqueous or aqueous-alcoholic gel. Thus, it may constitute only part or alternatively all of the aqueous phase. The aqueous phase may then optionally comprise, in addition to a synthetic phyllosilicate in gel form, one or more other gelling agents.

According to a particular embodiment, a synthetic phyllosilicate in the form of an aqueous gel that is suitable for use in the invention constitutes the aqueous phase.

It is a matter of routine operation for those skilled in the art to adjust the nature and the amount of the additives present in the compositions in accordance with the invention such that the desired cosmetic properties thereof are not thereby affected.

According to one embodiment, a composition of the invention may advantageously be in the form of a composition for caring for bodily or facial skin, in particular facial skin, or for nail care.

According to another embodiment, a composition of the invention may advantageously be in the form of a makeup base composition for making up bodily or facial skin, in particular facial skin, or for making up the nails.

According to another embodiment, a composition of the invention may advantageously be in the form of a foundation.

According to one embodiment, a composition of the invention may advantageously be in the form of a composition for making up the skin and especially the face. It may thus be an eyeshadow or a face powder.

The compositions according to the invention may moreover be packaged in pressurized form in an aerosol device or in a pump bottle; packaged in a device equipped with an openwork wall, especially a grille; packaged in a device equipped with a ball applicator (roll-on). In this regard, they contain the ingredients generally used in products of this type, which are well known to those skilled in the art.

The compositions packaged in aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

Such compositions are in particular prepared according to the general knowledge of those skilled in the art.

Throughout the description, including the claims, the term “comprising a” should be understood as being synonymous with “comprising at least one”, unless otherwise specified.

The terms “between . . . and . . . ” and “ranging from . . . to . . . ” should be understood as being inclusive of the limits, unless otherwise specified.

In the description and the examples, the percentages are percentages by weight. The ingredients are mixed in the order and under the conditions that are easily determined by those skilled in the art.

Methodology for the Oscillating Dynamic Rheology Measurements

These are harmonic-regime rheology measurements for measuring the elastic modulus.

The measurements are taken using a Haake RS150 (RheoStress RS150) rheometer on a product at rest, at 25° C. with a cone-plate geometry. The rotor diameter is Ø 60 mm. The gap is 0.103 mm. The cone geometry is C60/2°, for instance TiL L12028.

The harmonic-regime measurements make it possible to characterize the viscoelastic properties of the products. The technique consists in subjecting a material to a stress which varies sinusoidally over time and in measuring the response of the material to this stress. In a range in which the behavior is linear viscoelastic behavior (zone in which the strain is proportional to the stress), the stress (τ) and the strain (γ) are two sinusoidal functions of time which are written in the following manner:

τ(t)=τ₀ sin(ωt)

γ(t)=γ₀ sin(ωt+δ)

in which:

τ₀ represents the maximum amplitude of the stress (Pa);

γ₀ represents the maximum amplitude of the strain (−);

ω=2ΠN represents the angular frequency (rad·s⁻¹) with N representing the frequency (Hz); and

δ represents the phase shift of the stress relative to the strain (rad).

Thus, the two functions have the same angular frequency, but they are phase shifted by an angle δ. Depending on the phase shift δ between τ(t) and γ(t), the behavior of the system may be apprehended:
if δ=0, the material is purely elastic;
if δ=Π/2, the material is purely viscous (Newtonian fluid); and
if 0<δ<Π/2, the material is viscoelastic.

In general, the stress and the strain are written in complex form:

τ*(t)=τ₀e^{iωt}

γ*(t)=γ₀e^{(iωt+δ)}

A complex stiffness modulus, representing the overall resistance of the material to the strain, whether it is of elastic or viscous origin, is then defined by:

G*=τ*/γ*=G′+iG″

in which:

G′ is the storage modulus or elastic modulus, which characterizes the energy stored and totally restituted during a cycle, G′=(τ₀/γ₀)cos δ; and

G″ is the loss modulus or viscous modulus, which characterizes the energy dissipated by internal friction during a cycle, G″=(τ₀/γ₀)sin δ.

The parameter retained is the mean stiffness modulus G* recorded at the plateau measured at a frequency of 1 Hz.

The imposed-stress sweep is from 0.1 Pa to 2000 Pa and the measurement of the elastic modulus G′ is taken at small stresses when the structure of the material is not modified so as to obtain a value of the elastic modulus of the material at rest.

### FIGURES

FIG. 1 demonstrates the sol-gel transition by representation of the mean elastic modulus G′ at the plateau of compositions comprising a synthetic phyllosilicate that is suitable for use in the invention, in Pa measured at a temperature of 25° C. at a frequency of 1 Hz and at a stress from 0.1 Pa to 2000 Pa as a function of the concentration of synthetic phyllosilicate expressed as a weight percentage of active material.

FIG. 2 demonstrates the sol-gel transition by representation of the mean elastic modulus G′ at the plateau of compositions comprising a synthetic phyllosilicate that is suitable for use in the invention and 15% by weight of active material of an aluminum chlorohydrate, in Pa measured at a temperature of 25° C. at a frequency of 1 Hz and at a stress from 0.1 Pa to 2000 Pa as a function of the concentration of synthetic phyllosilicate expressed as a weight percentage of active material.

### EXAMPLES

### Example 1: Preparation of a Synthetic Phyllosilicate in Aqueous Gel Form that is Suitable for Use in the Invention

A synthetic phyllosilicate in aqueous gel form that is suitable for use in the invention is prepared according to the technology described in example 1 of patent application FR 2 977 580 from page 21, line 26 to page 22, line 29.

It was thus performed up to the formation of the hydrogel without the drying and lyophilization step.

Analysis of the X-ray diffractogram was performed with the aid of the materials and method used for the X-ray diffraction analyses that are detailed in patent application FR 2 977 580.

A characteristic diffraction line at 9.77 Å is observed.

The compositions according to the invention illustrated in the examples that follow comprise a synthetic phyllosilicate in accordance with the invention as obtained in this example 1.

### Example 2: Rheological Measurements—Demonstration of the Sol-Gel Transition of a Synthetic Phyllosilicate According to the Invention as Described in Example 1 in the Absence and in the Presence of Aluminum Chlorohydrate

The rheological measurements and especially the determination of the elastic modulus G′ are performed as indicated above.

In order to demonstrate the sol-gel transition, two series of tests were performed according to the rheological measurement protocol detailed above.

The first series of tests consists in testing compositions comprising synthetic phyllosilicate in gel form that is suitable for use in the invention and prepared according to example 1 in the absence of electrolyte and/or polyelectrolyte.

The second series of tests consists in testing compositions comprising synthetic phyllosilicate in gel form that is suitable for use in the invention and prepared according to example 1 in the presence of an aluminum salt and more precisely in the presence of 15% by weight of aluminum chlorohydrate.

For each of these series, different concentrations of synthetic phyllosilicate in gel form that is suitable for use in the invention were used, namely:
for the first series: 1%, 2%, 3%, 4%, 7%, 8% and 9.5% by weight; and
for the second series: 1%, 2%, 3% and 4% by weight.

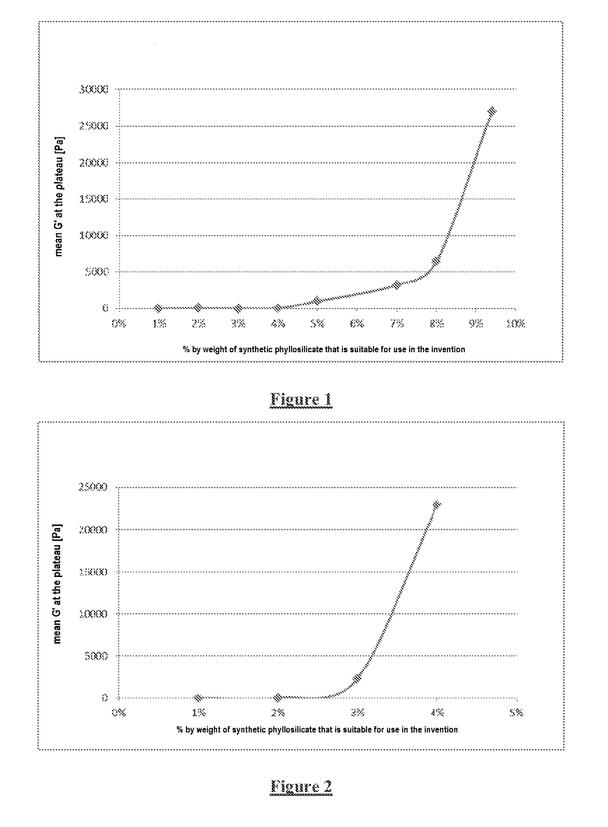

The results for each of the two series of tests are illustrated in FIGS. 1 and 2.

It is observed that passing from the liquid state to the gel state takes place at between 4% and 5% by weight of synthetic phyllosilicate active material when the latter is used alone in gel form, whereas the gel state is reached for a lower amount, namely between 2% and 3% by weight of synthetic phyllosilicate active material, when it is combined with aluminum chlorohydrate which is present in an amount of 15% by weight of active material relative to the total weight of the composition.

Thus, it is demonstrated that the combination of a gel of synthetic phyllosilicate that is suitable for use in the invention with an aluminum salt makes it possible to obtain gels with a lower content of synthetic phyllosilicate.

### Example 3: Rheological Measurements and Stability of the Compositions According to the Invention

Four compositions according to the invention (see table 1) comprising aluminum chlorohydrate as electrolyte are tested.

The measurement of the elastic modulus G′ is performed as detailed above.

The stability of the four compositions is evaluated at room temperature (20-30° C.), first after 24 hours, and then second after 2 months. The stability evaluation is performed by observation with the naked eye.

TABLE 1

| | Composition | | Composition | |
|---|---|---|---|---|
| | 3.1 | Composition | 3.3 according | Composition 3.4 |
| | according to | 3.2 according | to the | according to the |
| | the invention | to the invention | invention | invention |
| Ingredients | (weight %) | (weight %) | (weight %) | (weight %) |
| Synthetic | 1 | 2 | 3 | 4 |
| phyllosilicate (% | | | | |
| AM*) obtained | | | | |
| according to | | | | |
| example 1 | | | | |
| Water | qs | qs | qs | qs |
| Phenoxyethanol | 0.7 | 0.7 | 0.7 | 0.7 |
| (Neolone ™ PH | | | | |
| 100 Preservative | | | | |
| from the company | | | | |
| Dow Chemical | | | | |
| Company) | | | | |
| Aluminum | 30 (15% AM*) | 30 (15% AM*) | 30 (15% | 30 (15% AM*) |
| chlorohydrate | | | AM*) | |
| (Chlorhydrol 50 ® | | | | |
| from the company | | | | |
| Summitreheis) | | | | |
| G′ (Pa) | 0.5 | 12 | 2400 | 23000 |
| 24 hr stability | Stable | Stable | Stable | Stable |
| 2 month stability | Stable | Stable | Stable | Stable |

AM*: active material

Thus, it is observed that compositions 3.1 to 3.4 according to the invention remain stable not only after 24 hours, or alternatively after 2 months at room temperature.

Moreover, it is demonstrated that, within a composition according to the invention, the sol-gel transition takes place at the same concentrations of synthetic phyllosilicate as for the compositions illustrated in example 2, i.e. when the amount of synthetic phyllosilicate in accordance with the invention is between 2% and 3% by weight of active material for an amount of aluminum chlorohydrate of 15% by weight of active material relative to the total weight of the composition. Specifically, as indicated in table 1 above, the elastic modulus G′ varies, respectively, from 12 Pa to 2400 Pa.

### Example 4: Rheological Measurements of Compositions According to the Invention and of Comparative Compositions

### a Compositions Tested

One composition (composition 4.1) and three comparative compositions (compositions 4.2, 4.3 and 4.4) are tested.

In this example, the polyelectrolyte is illustrated by sodium hyaluronate (compositions 4.1, 4.2 and 4.4).

The synthetic phyllosilicate in powder form included in comparative composition 4.4 is prepared from the synthetic phyllosilicate in aqueous gel form obtained according to example 1, which is subjected to a drying step and then to a milling step.

| | Composition 4.1 | | Comparative | |
|---|---|---|---|---|
| | according to the | Comparative | composition 4.3 | |
| | invention | composition 4.2 | (weight % of | Comparative |
| | (weight % of active | (weight % of | active | composition 4.4 |
| | material) | active material) | material) | (weight % of |
| Ingredients | Aqueous gel | Aqueous gel | Aqueous gel | active material) |
| Water | 94 | 95 | 95 | 94 |
| Synthetic | 5 | — | 5 | — |
| phyllosilicate in | | | | |
| aqueous gel form | | | | |
| obtained according | | | | |
| to example 1 | | | | |
| Sodium | 1 | 1 | — | 1 |
| hyaluronate | | | | |
| (Hyactive ® 120 | | | | |
| from the company | | | | |
| Contripo) | | | | |
| Synthetic | — | — | — | 5 |
| phyllosilicate in | | | | |
| powder form | | | | |

### b Macroscopic Appearance

Each composition is evaluated in its jar.

| | Composition 4.1 | | | |
|---|---|---|---|---|
| | according to the | Comparative | Comparative | Comparative |
| | invention | composition 4.2 | composition 4.3 | composition 4.4 |
| Macroscopic | texture which does | liquid | flows at rest | fluid; the |
| appearance | not flow under its | | (flows under its | synthetic |
| | own weight | | own weight) | phyllosilicate in |
| | | | | powder form |
| | | | | decants |

Only composition 4.1 according to the invention has a texture that does not flow under its own weight.

Thus, a synthetic phyllosilicate according to the invention in gel form is not destructured by the addition of a polyelectrolyte such as sodium hyaluronate and even contributes toward increasing the viscosity of compositions comprising such a polyelectrolyte. This aspect is corroborated by the rheological characterizations featured below.

Moreover, a synthetic phyllosilicate in powder form does not make it possible to obtain the expected result especially in terms of viscosity, gelling effect, thickening effect, stability and homogeneity of the deposit.

### c Rheological Measurements

Composition 4.1 according to the invention and the comparative compositions 4.2 and 4.3 were characterized in oscillating rheology according to the materials and methods detailed above, which makes it possible to evaluate the viscous moduli and elastic moduli.

The liquid or solid nature is determined by the delta value as a function of the applied stress.

| Rheological | | | |
|---|---|---|---|
| magnitudes | | | |
| For a stress of 1 Pa | Composition 4.1 | | |
| and a frequency of | according to the | Comparative | Comparative |
| 1 Hz | invention | composition 4.2 | composition 4.3 |
| Elastic modulus G′ | 716 ± 50 | 0.021 ± 0.004 | 280 ± −20 |
| at the curve plateau | | | |
| Viscous modulus | 70 ± 4 | 0.135 ± −0.007 | 21 ± −4 |
| G″ | | | |
| at the curve plateau | | | |
| Delta (δ) | 6 ± −1 | 81 ± 2 | 4 ± 1 |
| CONCLUSION | The elastic modulus | The product is liquid | The elastic modulus G′ |
| | G′ value is markedly | | is higher than the viscous |
| | higher than that for | | modulus G″ |
| | the sodium | | |
| | hyaluronate gel | | |
| | alone or for the gel | | |
| | containing 5% | | |
| | synthetic | | |
| | phyllosilicate alone. | | |

Composition 4.1 according to the invention has an elastic modulus G′ value that is markedly higher than that for the aqueous sodium hyaluronate gel alone (comparative composition 4.2) or for the aqueous gel containing 5% of synthetic phyllosilicate alone (comparative composition 4.3).

The delta values also show that the aqueous gel containing 1% of sodium hyaluronate (comparative composition 4.2) has “liquid” behavior at rest (delta>45°), whereas the aqueous gel containing 5% of synthetic phyllosilicate in gel form has “solid” behavior (delta<45°).

Consequently, solutions of active agents that are difficult to formulate such as electrolytes or polyelectrolytes may be gelled by means of the synthetic phyllosilicate used in gel form, which facilitates the application to the skin and the pleasantness of the cosmetic product obtained.

### Example 5: Cream of Oil/Water Emulsion Type Based on a Synthetic Phyllosilicate in Gel Form

Procedure:

Once the preserving system has been dissolved in water (at the required temperature), the hydrophilic gelling agent is added while stirring with a Rayneri blender at about 70° C. until the gel has homogenized, and the active agent 3-hydroxy-2-pentylcyclopentaneacetic acid is added. The fatty phase was homogenized (at the temperature required to obtain a homogeneous liquid phase). When the mixtures of the two phases were homogeneous, the emulsion was formed by adding the fatty phase to the aqueous phase with stirring using a Moritz blender. The emulsion was cooled with stirring using a Rayneri blender until a homogeneous smooth cream was obtained.

| | | COMPOSITION |
|---|---|---|
| | | 5 according to |
| | INCI Name/Ingredients | the invention |
| Fatty | GLYCERYL STEARATE (and) | 2 |
| phase | PEG-100 STEARATE | |
| | HYDROGENATED POLYISOBUTENE | 12 |
| Aqueous | PRESERVING AGENT | qs |
| phase | WATER | qs 100 |
| | Dipropylene glycol | 1% |
| | 3-Hydroxy-2- | 1.6% |
| | pentylcyclopentaneacetic acid | |
| | Synthetic phyllosilicate (gel) | 30 |
| | (10.3% AM) | |

### Macroscopic Appearance:

Composition 5 according to the invention has a stable and homogeneous texture at 24 hr, which does not flow under its own weight.

Thus, a synthetic phyllosilicate according to the invention in gel form is not destructured by the addition of a polyelectrolyte such as 3-hydroxy-2-pentylcyclopentaneacetic acid.

### Example 6: Comparison of the Infrared Spectrum Between Natural Talc and the Synthetic Phyllosilicate that is Suitable for Use in the Invention in an Antiperspirant Composition

| | COMPOSITION | COMPOSITION |
|---|---|---|
| | 6.1 | 6.2 |
| | according to the | outside the |
| INCI Name/Ingredients | invention | invention |
| Aluminum chlorohydrate | 30 | 30 |
| (50% solution - Cluron) | | |
| Synthetic phyllosilicate (gel) | 24.4 | — |
| Natural talc | — | 24.4 |
| Dimethicone 10 cSt | 2.5 | 12 |
| Dicaprylyl Ether | 2.5 | 2.5 |
| PRESERVING AGENT | 0.7% | 0.7% |
| WATER | qs 100 | qs 100 |

The infrared spectrum of compositions 6.1 and 6.2 was measured.

The machine used was a Nicolet 6700 FTIR Fourier transform spectrometer, equipped with an integration sphere, with an InGaA detector and a CaF₂ separator and a resolution of 4 cm⁻¹. In other words, the values of the absorption bands given in this description are to be considered as being more or less 2 cm⁻¹.

The near infrared recordings of the stretching region located at 7185 cm⁻¹ were broken down by pseudo-Voigt functions using the Fityk software (Wojdyr, 2010).

The compositions were heated to 120° C. and 400° C.

Stretching amplifications of plus or minus 200 cm⁻¹ on either side of a suspected absorption band were performed.

| | 7156 cm⁻¹ | 7186 cm⁻¹ | 7200 cm⁻¹ |
|---|---|---|---|
| Composition | 2v Mg₂FeOH | 2v Mg₃OH | Si—OH silanols |
| Composition 6.1 with | No | Yes | Yes |
| synthetic | | | |
| phyllosilicate | | | |
| (invention) | | | |
| Composition 6.2 with | Yes | Yes | No |
| natural talc | | | |
| (outside the | | | |
| invention) | | | |

### Example 7: Compositions Based on Sodium Chloride as Electrolyte

Examples 7.1 to 7.4 were prepared by simple mixing of the ingredients with stirring using a Rayneri blender at room temperature (20-30° C.) until a homogeneous mixture was formed.

Compositions 7.2 to 7.4 according to the invention and comparative composition 7.1 were characterized in oscillating rheology according to the materials and method detailed above in the description, which makes it possible to evaluate the consistency values G*.

| | | Ex. 7.2 | Ex. 7.3 | Ex. 7.4 |
|---|---|---|---|---|
| | Ex. 7.1 | according to | according to | according to |
| INCI Name/ | outside the | the | the | the |
| Ingredients | invention | invention | invention | invention |
| Synthetic | 29 | 29 | 29 | 29 |
| phyllosilicate | | | | |
| (gel) | | | | |
| containing | | | | |
| 10.3% dry | | | | |
| matter | | | | |
| NaCl | 0 | 1 | 2 | 4 |
| WATER | qs 100 | qs 100 | qs 100 | qs 100 |
| Consistency | flows under | Gel does not | Gel does not | Gel does not |
| | its own | flow under | flow under | flow under |
| | weight | its own | its own | its own |
| | | weight | weight | weight |
| Consistency | | >> Ex7.1 | > Ex7.2 | > Ex7.3 |
| comparison | | | | |
| G* | | | | |

It was observed that by adding sodium chloride, a homogeneous gel was formed which does not flow under its own weight and of which the consistency increases as the concentration in the tested concentration range increases.

### Example 8: Compositions Based on 3-hydroxy-2-pentylcyclopentylacetic acid as Electrolyte

Examples 8.1 to 8.4 were prepared by simple mixing of the ingredients with stirring using a Rayneri blender at room temperature (20-30° C.) until a homogeneous mixture was formed.

Compositions 8.2 to 8.4 according to the invention and comparative composition 8.1 were characterized in oscillating rheology according to the materials and method detailed above in the description, which makes it possible to evaluate the consistency values G*.

| | | Ex. 8.2 | Ex. 8.3 | |
|---|---|---|---|---|
| | Ex. 8.1 | according to | according to | Ex. 8.4 |
| | outside the | the | the | according |
| INCI Name/Ingredients | invention | invention | invention | to the invention |
| Synthetic phyllosilicate | 29 | 29 | 29 | 29 |
| (gel) containing 10.3% dry | | | | |
| matter | | | | |
| 3-Hydroxy-2- | 0 | 1 | 2 | 4 |
| pentylcyclopentaneacetic | | | | |
| acid | | | | |
| WATER | qs 100 | qs 100 | qs 100 | qs 100 |
| Consistency | flows under | Gel does not | Gel does not | Gel does not |
| | its own | flow under | flow under | flow under |
| | weight | its own | its own | its own |
| | | weight | weight | weight |
| Consistency comparison G* | | >>> Ex 8.1 | > Ex 8.2 | >> Ex 8.3 |

It was observed that by adding 3-hydroxy-2-pentylcyclopentaneacetic acid a homogeneous gel was formed which does not flow under its own weight and of which the consistency increases as the concentration in the tested concentration range increases.

### Example 9: Other Compositions According to the Invention Based on Aluminum Chlorohydrate and Compositions Outside the Invention

Characterizations

Besides the usual characterizations such as the stability of the systems, the deposits obtained by applying the formulations to a glass plate are evaluated on criteria of drying time, film quality, water resistance and friction resistance.

Deposit: spread 2 cm wide and 100 μm thick on a glass plate. The deposit is produced using a film spreader, and is left to dry for 2 hours and 24 hours.

Quality of the deposit: the presence of cracks is observed

Friction resistance: the film is scratched in a to-and-fro motion with a spatula.

Water resistance: a drop of water is deposited on the film and the spreading of the drop over the deposit is observed (will be completed by quantitative measurements)

The formulations are evaluated on the following criteria:

### A) Friction Resistance

+: good resistance. The film breaks down slowly.
++: very good resistance. The film breaks down little.
−: poor resistance. The film breaks down rapidly.
−−: very poor resistance. The film breaks down immediately.

### B) Water Resistance

+: good resistance. The drop spreads slowly.
++: very good resistance. The drop does not spread.
−: poor resistance. The drop spreads rapidly.
−−: very poor resistance. The drop spreads immediately (wider spreading surface).

9.1: Composition in Gel Form

| INGREDIENTS | Composition 1 | Composition 2 |
|---|---|---|
| Synthetic phyllosilicate | 48.78 (4% AM*) | 48.78 (4% AM*) |
| prepared according to example 1 | | |
| (Gel) | | |
| Demineralized water | qs | qs |
| Phenoxyethanol | 0.7 | 0.7 |
| (Neolone ™ PH 100 Preserver | | |
| from the company Dow | | |
| Chemical Company) | | |
| Aluminum chlorohydrate | 30 | 30 |
| (Chlorhydrol ® 50 from the | | |
| company Summitreheis) | | |
| Acrylate copolymer | 5 | 5 |
| (Daitosol ® 5000 AD from the | | |
| company Daito Kasei Kogyo) | | |
| (% AM) | | |
| Hydroxypropyl starch phosphate | — | 2.5 |
| Structure XL ® | | |
| from the company AkzoNobel | | |
| 24 hr stability | Yes | Yes |
| Drying at 2 hr | Yes | Yes |
| Presence of cracks | Yes | Yes |
| Friction resistance | ++ | ++ |
| Water resistance | + | + |

% AM*: weight percentage of synthetic phyllosilicate active material in water

Compositions 1 and 2 in gel form are stable and make it possible to obtain a good-quality film and a dry feel, and to give good water resistance and friction resistance. These compositions conserve a pleasant sensory perception suitable for an antiperspirant/deodorant application.

9.2 Compositions in Emulsion Form

| | | | Composition | |
|---|---|---|---|---|
| | Composition | Composition | 5 according | Composition 6 |
| | 3 outside the | 4 outside the | to the | according to |
| INGREDIENTS | invention | invention | invention | the invention |
| Synthetic phyllosilicate | — | — | 24.39 (2% | 24.39 (2% |
| prepared according to | | | AM*) | AM*) |
| example 1 (Gel) | | | | |
| Ceteareth 33 | 1.25 | 1.25 | — | — |
| Cetearyl alcohol | 2.5 | 1.25 | — | — |
| PDMS 350 cSt | 0.50 | — | — | — |
| (Polydimethylsiloxane) | | | | |
| (Element 14 PDMS 10- | | | | |
| A ™ from the company | | | | |
| Momentive Performance | | | | |
| Materials Inc.) | | | | |
| Isododecane | — | 5 | 5 | 5 |
| (Isododecane ®) | | | | |
| Demineralized water | qs | qs | qs | qs |
| Phenoxyethanol | 0.70 | 0.70 | 0.70 | 0.70 |
| (Neolone ™ PH 100 | | | | |
| Preserver from the | | | | |
| company Dow Chemical | | | | |
| Company) | | | | |
| Aluminum chlorohydrate | 30.00 | 30.00 | 30.00 | 30.00 |
| (Chlorhydrol ® 50 from | | | | |
| the company | | | | |
| Summitreheis) | | | | |
| Acrylic acid/isobutyl | — | 5.00 | 5.00 | — |
| acrylate/isobornyl acrylate | | | | |
| copolymer (Mexomer | | | | |
| PAS ® sold by the | | | | |
| company Chimex) | | | | |
| Hydroxypropyl starch | — | — | 2.50 | 2.50 |
| phosphate | | | | |
| Structure XL | | | | |
| from the company | | | | |
| AkzoNobel | | | | |
| 24 hr stability | ok | ok | ok | ok |
| Drying at 2 hr | No | No | Yes | Yes |
| Drying at 24 hr | Yes | Yes | Yes | Yes |
| Presence of cracks | No | No | No | Yes |
| Water resistance | − | − | ++ | + |

% AM*: weight percentage of synthetic phyllosilicate active material in water

| | Composition 3 | Composition 7 | Composition 8 | Composition 9 |
|---|---|---|---|---|
| | outside the | outside the | outside the | according to the |
| INGREDIENTS | invention | invention | invention | invention |
| Synthetic phyllosilicate | — | — | — | 24.39 (2% AM*) |
| prepared according to example | | | | |
| 1 (Gel) | | | | |
| Ceteareth 33 | 1.25 | — | 1.25 | — |
| Cetearyl alcohol | 2.50 | — | 2.50 | — |
| PDMS 350 cSt | 0.50 | — | 0.50 | 0.50 |
| (Polydimethylsilaxane) | | | | |
| (Element 14 PDMS 10-A ™ | | | | |
| from the company Momentive | | | | |
| Performance Materials Inc.) | | | | |
| Demineralized water | qs | qs | qs | qs |
| Phenoxyethanol | 0.70 | — | 0.70 | 0.70 |
| (Neolone ™ PH 100 Preserver | | | | |
| from the company Dow | | | | |
| Chemical Company) | | | | |
| Aluminum chlorohydrate | 30.00 | — | 30.00 | 30.00 |
| (Chlorhydrol ® 50 from the | | | | |
| company Summitreheis) | | | | |
| Styrene/acrylates/ammonium | — | 40.00 | 10.00 | 10.00 |
| methacrylate copolymer | | | | |
| (Syntran 5760 ® from the | | | | |
| company Interpolymer) | | | | |
| 24 hr stability | ok | ok | ok | ok |
| Drying at 2 hr | No | No | Yes | Yes |
| Drying 24 hr | Yes | Yes | Yes | Yes |
| Presence of cracks | No | No | No | No |
| Friction resistance | − | ++ | + | ++ |
| Water resistance | − | ++ | + | ++ |

The combination of a synthetic phyllosilicate that is suitable for use in the invention and of the film-forming agent makes it possible to obtain better properties in terms of resistance of the film to water and to friction, and better drying times than for a formulation containing the synthetic phyllosilicate alone and also much better properties than for a formulation without phyllosilicate and without film-forming agent.

### Example 10: Other Compositions According to the Invention Based on Deodorant Active Agents Such as Zinc Pidolate, Sodium (1-oxododecyl)-L-ethyl arginate hydroxychloride or Potassium Alum as Electrolyte

Examples 10.1 to 10.4 were prepared by simple mixing of the ingredients with magnetic stirring at room temperature (20-30° C.) until a homogeneous mixture was formed.

| | Composition | Composition | Composition | Composition |
|---|---|---|---|---|
| | 10.1 | 10.2 | 10.3 | 10.4 |
| | according to the | according to the | according to the | according to the |
| | invention | invention | invention | invention |
| Ingredients | (weight %) | (weight %) | (weight %) | (weight %) |
| Synthetic | 3 | 3 | 3 | 3 |
| phyllosilicate (% | | | | |
| AM*) obtained | | | | |
| according to | | | | |
| example 1 | | | | |
| Water | qs | qs | qs | qs |
| Phenoxyethanol | 0.7 | 0.7 | 0.7 | 0.7 |
| (Neolone ™ PH | | | | |
| 100 Preservative | | | | |
| from the | | | | |
| company Dow | | | | |
| Chemical | | | | |
| Company) | | | | |
| Zinc | 1 | | | |
| pyrrolidonecarboxylate | | | | |
| (or zinc pidolate) | | | | |
| (Zincidone ® from the | | | | |
| company UCIB | | | | |
| Solabia) | | | | |
| Sodium (1- | | 2 | | |
| oxododecyl)-L-ethyl | | | | |
| arginate hydrochloride | | | | |
| dissolved in glycerol | | | | |
| (Aminat G ® from the | | | | |
| company Vedeqsa) | | | | |
| Zinc gluconate | | | 1 | |
| (from the company | | | | |
| Soproreal) | | | | |
| Potassium alum | | | | 2 |
| (Potassium Alum ® | | | | |
| from the company | | | | |
| Holland) | | | | |
| 24 hr stability | Stable | ND | ND | Stable |

AM*: active material
ND: not determined

Thus, it is observed that compositions 10.1 and 10.4 according to the invention are stable after 24 hours. It was also observed that a synthetic phyllosilicate according to the invention is not destructured by the addition of electrolytes corresponding to deodorant active agents such as zinc pidolate, sodium (1-oxododecyl)-L-ethyl arginate hydroxychloride or potassium alum, and even contributes toward increasing the viscosity of compositions comprising such electrolytes.

1. A composition comprising:
(a) at least one synthetic phyllosilicate of molecular formula Mg₃Si₄O₁₀(OH)₂ in the form of an aqueous or aqueous-alcoholic gel; and
(b) at least one electrolyte and/or at least one polyelectrolyte.

2. The composition as claimed in claim 1, wherein the composition has an X-ray diffraction line greater than 9.4 Å and less than or equal to 9.8 Å.

3. The composition as claimed in claim 1, wherein the composition has an infrared absorption band at 7200 cm⁻¹ corresponding to the stretching vibration attributed to the silanol groups Si—OH at the edge of the phyllosilicate leaflets.

4. The composition as claimed in claim 1, wherein there is no infrared absorption band at 7156 cm⁻¹.

5. The composition as claimed in claim 1, wherein the synthetic phyllosilicate in the form of an aqueous gel constitutes the aqueous phase.

6. The composition as claimed in claim 1, in wherein said synthetic phyllosilicate is present in an amount ranging from 0.01% to 20% by weight relative to the total weight of the composition.

7. The composition as claimed in claim 1, wherein the synthetic phyllosilicate in the form of an aqueous or aqueous-alcoholic gel is present in an amount ranging from 0.5% to 20% by weight of active material relative to the total weight of the aqueous phase.

8. The composition as claimed in claim 1, wherein the electrolyte is chosen from salts of the cucurbic acid derivatives corresponding to formula (I) below: 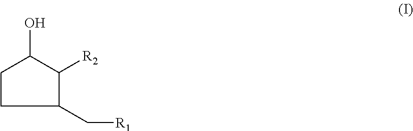
wherein:
R₁ represents a radical COOR₃, R₃ denoting a hydrogen atom or a C₁-C₄ alkyl radical;
R₂ represents a saturated or unsaturated linear hydrocarbon-based radical containing from 1 to 18 carbon atoms or a saturated or unsaturated branched or cyclic hydrocarbon-based radical containing from 3 to 18 carbon atoms;
and also the optical isomers thereof, and corresponding salts.

9. The composition as claimed in claim 1, wherein the electrolyte is chosen from lipophilic salicylic acid derivatives corresponding to formula (II) 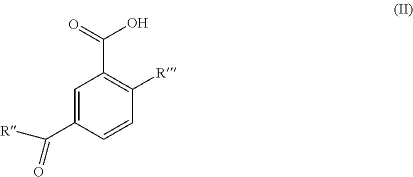
wherein:
the radical R″ denotes a linear, branched or cyclic saturated aliphatic chain containing from 2 to 22 carbon atoms; an unsaturated chain containing from 2 to 22 carbon atoms containing one or more double bonds; an aromatic nucleus linked to the carbonyl radical directly or via saturated or unsaturated aliphatic chains containing from 2 to 7 carbon atoms;
R′″ is a hydroxyl group or an ester group of formula: 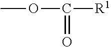
in which R¹ denotes a saturated or unsaturated, linear or branched aliphatic chain containing from 1 to 18 carbon atoms;
and also salts thereof derived from a mineral or organic base.

10. The composition as claimed in claim 1, wherein the electrolyte is chosen from an aluminum and/or zirconium salt or complex, a silver salt, a zinc salt, and mixtures thereof.

11. The composition as claimed in claim 1, wherein the electrolyte is chosen from an aluminum and/or zirconium salt, an aluminum and/or zirconium complex, and mixtures thereof, and also comprising (c) at least one film-forming polymer chosen from a hydrophilic film-forming polymer and a hydrophobic film-forming polymer, and mixtures thereof.

12. The composition as claimed in claim 1, wherein the polyelectrolyte is chosen from:
anionic polysaccharides,
cationic polymers including chitosans and polymeric quaternary ammonium salts,
crosslinked and/or neutralized 2-methyl-2-[(1-oxo-2-propenyl)amino]-1-propanesulfonic acid homopolymers and copolymers,
acrylic homopolymers and copolymers,
polyacrylic acids,
polyacrylic acid/alkyl acrylate copolymers,
and mixtures thereof.

13. The composition as claimed in claim 1, wherein the polyelectrolyte is chosen from a crosslinked hyaluronic acid, a non-crosslinked hyaluronic acid, salts thereof and mixtures thereof.

14. The composition as claimed in claim 1, wherein the electrolyte and/or the polyelectrolyte are present in a total amount ranging from 0.1% to 30% by weight of active material relative to the total weight of the composition.

15. The composition as claimed in claim 11, wherein the hydrophilic film-forming polymer is chosen from polyurethanes, vinyl polymers, natural polymers, latices and pseudolatices, and mixtures thereof.

16. The composition as claimed in claim 11, wherein the hydrophobic film-forming polymer is chosen from:
(i) polymers of interpenetrating polymer network type;
(ii) grafted silicone polymers;
(iii) non-neutralized (meth)acrylic acid/N-tert-butylacrylamide copolymers;
(iv) non-neutralized crotonic acid/vinyl acetate copolymers;
(v) tetrapolymers of (meth)acrylic acid, of (meth)acrylates and of C₈-C₂₄ alkyl (meth)acrylate;
(vi) film-forming pseudoblocks;
(vii) hydrocarbon-based block copolymers; and
(viii) mixtures thereof.

17. The composition as claimed in claim 11 wherein the film-forming polymer is present in an amount ranging from 0.1% to 40% by weight of active material relative to the total weight of the composition.

18. The composition as claimed in claim 1, wherein the composition is cosmetic or dermatological and comprises a physiologically acceptable medium.

19. A cosmetic process for treating the skin and/or the nails, comprising at least the step of applying to the skin and/or the nails a composition as claimed in claim 1.

20. A cosmetic process for treating perspiration consisting in the step of applying to the surface of the skin a composition as claimed in claim 1.
